(19) United States
(12) Patent Application Publication
(10) Pub. No.: US 2024/0336599 A1
(43) Pub. Date: Oct. 10, 2024

(54) PHOTOSENSITIVE POLYAMIC ACID AND DIAZIRINE COMPOSITIONS

(71) Applicant: PROMERUS, LLC, AKRON, OH (US)

(72) Inventors: PRAMOD KANDANARACHCHI, AKRON, OH (US); LARRY F. RHODES, AKRON, OH (US)

(73) Assignee: PROMERUS, LLC, AKRON, OH (US)

(21) Appl. No.: 18/611,785

(22) Filed: Mar. 21, 2024

Related U.S. Application Data
(60) Provisional application No. 63/453,555, filed on Mar. 21, 2023.

(51) Int. Cl. C07D 403/14; C08G 73/10; C08K 5/3442; G03F 7/004; G03F 7/038

(52) U.S. Cl. CPC C07D 403/14; C08G 73/105; C08K 5/3442; G03F 7/0045; G03F 7/0387

(57) ABSTRACT

Embodiments in accordance with the present invention encompass a composition comprising a polyamic acid or an end capped polyamic acid and a diazirine, which are useful as photosensitive compositions for forming films that can be patterned to create structures for microelectronic devices, microelectronic packaging, microelectromechanical systems, optoelectronic devices and displays. In some embodiments the compositions of this invention are shown to feature excellent hitherto unachievable mechanical properties. The negative images formed therefrom exhibit improved thermo-mechanical properties, among other property enhancements.

### CROSS REFERENCE TO RELATED APPLICATIONS

This application claims the benefit of U.S. Provisional Application No. 63/453,555, filed Mar. 21, 2023, which is incorporated herein by reference in its entirety.

### BACKGROUND OF THE INVENTION

### Field of the Invention

The present invention relates to a series of photosensitive polyamic acid and diazirine compositions. More specifically, the present invention relates to a photosensitive composition containing polyamic acid or end capped polyamic acid compositions with a variety of diazirines. The compositions of this invention are free of any acrylate crosslinkers and may optionally contain one or more epoxy crosslinkers. The compositions of this invention are useful for forming microelectronic and/or optoelectronic devices and assemblies thereof, and more specifically, such compositions exhibit improved thermal, mechanical and opto-electronic properties.

### Description of the Art

Organic polymer materials are increasingly being used in the microelectronics and optoelectronics industries for a variety of applications. For example, the uses for such organic polymer materials include permanent interlevel dielectrics, redistribution layers (RDL), stress buffer layers, chip stacking and/or bonding, leveling or planarization layers, alpha-particle barriers, passivation layers, among others, in the fabrication of a variety of microelectronic and optoelectronic devices. Where such organic polymer materials are photosensitive, thus self-imageable, and therefore, offer additional advantage of reducing the number of processing steps required for the use of such layers and structures made therefrom. Additionally, such organic polymer materials enable the direct adhesive bonding of devices and device components to form various structures. Such devices include microelectromechanical systems (MEMS), microoptoelectromechanical systems (MOEMS) and the semiconductor device encompassing a complementary metal oxide semiconductor (CMOS) image sensor dam structure, and the like.

There has been innumerable polymeric materials used in the art in order to achieve the above noted desired requirements. One such class of polymers include polyimides and its precursor, polyamic acid. However, most of the polyimides and polyamic acids disclosed in the art are generally used for positive tone image forming films, and may not be suitable for many applications. Some of the drawbacks include use of highly toxic and corrosive phenolic monomers which provide alkali solubility that is required for forming positive tone compositions. Other property disadvantages include insolubility of the polyimides and/or the precursor polyamic acids in commonly used solvents in the electronic industry, poor photo imaging capabilities, among others. Even more importantly, such compositions suffer from poor thermo-mechanical properties and may require high cure temperatures, often times higher than 300° C., which are undesirable. See for example, U.S. Pat. Nos. 8,946,852 B2 and 7,485,405 B2.

In addition, conventional polyimide and/or polyamic acid compositions may contain fugitive volatile acrylic based crosslinkers, which pose environmental issues releasing volatile toxic components to the atmosphere. Accordingly, there is a greater need for developing environmentally friendly materials.

Accordingly, it is an object of this invention to provide a composition containing a series of polyamic acid or end capped polyamic acid in combination with a variety of diazirines that provide improved thermo-mechanical properties.

It is also an object of this invention to provide compositions which can be cured at lower temperatures than the conventional polyimides that exhibit improved thermo-mechanical properties.

Other objects and further scope of the applicability of the present invention will become apparent from the detailed description that follows.

### SUMMARY OF THE INVENTION

Surprisingly, it has now been found that incorporating a variety of diazirines, such as a diazirine of formula (II) as described herein with a series of polyamic acid of formula (IA) or an end capped polyamic acid of formulae (IB) or (IC) as described herein in a photosensitive composition provides hitherto unattainable thermo-mechanical properties, among other property advantages. More specifically, the polyamic acid of formula (IA) or an end capped polyamic acid of formulae (IB) or (IC) as disclosed herein can be made by employing any of the known dianhydrides and diamines in combination with a suitable end capping agent, which are soluble in commonly used organic solvents. The polyamic acid of this invention can then be combined with a diazirine of formula (II) optionally in combination with one or more additives as disclosed herein to form photosensitive compositions which feature excellent thermo-mechanical properties, photo-imaging properties, low cure temperatures, generally below 100° C. or lower, among other property enhancements.

### BRIEF DESCRIPTION OF THE DRAWINGS

Embodiments in accordance with the present invention are described below with reference to the following accompanying figures and/or images. Where drawings are provided, it will be drawings which are simplified portions of various embodiments of this invention and are provided for illustrative purposes only.

FIG. 1 and FIG. 2 show top down optical micrograph images of a composition embodiment of this invention

### DETAILED DESCRIPTION OF THE INVENTION

As used herein, the articles “a,” “an,” and “the” include plural referents unless otherwise expressly and unequivocally limited to one referent.

Since all numbers, values and/or expressions referring to quantities of ingredients, reaction conditions, etc., used herein and in the claims appended hereto, are subject to the various uncertainties of measurement encountered in obtaining such values, unless otherwise indicated, all are to be understood as modified in all instances by the term “about.”

Where a numerical range is disclosed herein such range is continuous, inclusive of both the minimum and maximum values of the range as well as every value between such minimum and maximum values. Still further, where a range refers to integers, every integer between the minimum and maximum values of such range is included. In addition, where multiple ranges are provided to describe a feature or characteristic, such ranges can be combined. That is to say that, unless otherwise indicated, all ranges disclosed herein are to be understood to encompass any and all sub-ranges subsumed therein. For example, a stated range of from “1 to 10” should be considered to include any and all sub-ranges between the minimum value of 1 and the maximum value of 10. Exemplary sub-ranges of the range 1 to 10 include, but are not limited to, 1 to 6.1, 3.5 to 7.8, and 5.5 to 10, etc.

As used herein, the expression “alkyl” means a saturated, straight-chain or branched-chain hydrocarbon substituent having the specified number of carbon atoms. Particular alkyl groups are methyl, ethyl, n-propyl, isopropyl, tert-butyl, and so on. Derived expressions such as “alkoxy”, “thioalkyl”, “alkoxyalkyl”, “hydroxyalkyl”, “alkylcarbonyl”, “alkoxycarbonylalkyl”, “alkoxycarbonyl”, “diphenylalkyl”, “phenylalkyl”, “phenylcarboxyalkyl” and “phenoxyalkyl” are to be construed accordingly.

As used herein, the expression “cycloalkyl” includes all of the known cyclic groups. Representative examples of “cycloalkyl” includes without any limitation cyclopropyl, cyclobutyl, cyclopentyl, cyclohexyl, cycloheptyl, cyclooctyl, and the like. Derived expressions such as “cycloalkoxy”, “cycloalkylalkyl”, “cycloalkylaryl”, “cycloalkylcarbonyl” are to be construed accordingly.

As used herein, the expression “perhaloalkyl” represents the alkyl, as defined above, wherein all of the hydrogen atoms in said alkyl group are replaced with halogen atoms selected from fluorine, chlorine, bromine or iodine. Illustrative examples include trifluoromethyl, trichloromethyl, tribromomethyl, triiodomethyl, pentafluoroethyl, pentachloroethyl, pentabromoethyl, pentaiodoethyl, and straight-chained or branched heptafluoropropyl, heptachloropropyl, heptabromopropyl, nonafluorobutyl, nonachlorobutyl, undecafluoropentyl, undecachloropentyl, tridecafluorohexyl, tridecachlorohexyl, and the like. Derived expression, “perhaloalkoxy”, is to be construed accordingly. It should further be noted that certain of the alkyl groups as described herein, such as for example, “alkyl” may partially be fluorinated, that is, only portions of the hydrogen atoms in said alkyl group are replaced with fluorine atoms and shall be construed accordingly.

As used herein the expression “acyl” shall have the same meaning as “alkanoyl”, which can also be represented structurally as “R—CO—,” where R is an “alkyl” as defined herein having the specified number of carbon atoms. Additionally, “alkylcarbonyl” shall mean same as “acyl” as defined herein. Specifically, “(C₁-C₄)acyl” shall mean formyl, acetyl or ethanoyl, propanoyl, n-butanoyl, etc. Derived expressions such as “acyloxy” and “acyloxyalkyl” are to be construed accordingly.

As used herein, the expression “aryl” means substituted or unsubstituted phenyl or naphthyl. Specific examples of substituted phenyl or naphthyl include o-, p-, m-tolyl, 1,2-, 1,3-, 1,4-xylyl, 1-methylnaphthyl, 2-methylnaphthyl, etc. “Substituted phenyl” or “substituted naphthyl” also include any of the possible substituents as further defined herein or one known in the art.

As used herein, the expression “arylalkyl” means that the aryl as defined herein is further attached to alkyl as defined herein. Representative examples include benzyl, phenylethyl, 2-phenylpropyl, 1-naphthylmethyl, 2-naphthylmethyl and the like.

As used herein, the expression “alkenyl” means a non-cyclic, straight or branched hydrocarbon chain having the specified number of carbon atoms and containing at least one carbon-carbon double bond, and includes ethenyl and straight-chained or branched propenyl, butenyl, pentenyl, hexenyl, and the like. Derived expression, “arylalkenyl” and five membered or six membered “heteroarylalkenyl” is to be construed accordingly. Illustrative examples of such derived expressions include furan-2-ethenyl, phenylethenyl, 4-methoxyphenylethenyl, and the like.

As used herein, the expression “heteroaryl” includes all of the known heteroatom containing aromatic radicals. Representative 5-membered heteroaryl radicals include furanyl, thienyl or thiophenyl, pyrrolyl, isopyrrolyl, pyrazolyl, imidazolyl, oxazolyl, thiazolyl, isothiazolyl, and the like. Representative 6-membered heteroaryl radicals include pyridinyl, pyridazinyl, pyrimidinyl, pyrazinyl, triazinyl, and the like radicals. Representative examples of bicyclic heteroaryl radicals include, benzofuranyl, benzothiophenyl, indolyl, quinolinyl, isoquinolinyl, cinnolyl, benzimidazolyl, indazolyl, pyridofuranyl, pyridothienyl, and the like radicals.

As used herein, the expression “heterocycle” includes all of the known reduced heteroatom containing cyclic radicals. Representative 5-membered heterocycle radicals include tetrahydrofuranyl, tetrahydrothiophenyl, pyrrolidinyl, 2-thiazolinyl, tetrahydrothiazolyl, tetrahydrooxazolyl, and the like. Representative 6-membered heterocycle radicals include piperidinyl, piperazinyl, morpholinyl, thiomorpholinyl, and the like. Various other heterocycle radicals include, without limitation, aziridinyl, azepanyl, diazepanyl, diazabicyclo[2.2.1]hept-2-yl, and triazocanyl, and the like.

“Halogen” or “halo” means chloro, fluoro, bromo, and iodo.

In a broad sense, the term “substituted” is contemplated to include all permissible substituents of organic compounds. In a few of the specific embodiments as disclosed herein, the term “substituted” means substituted with one or more substituents independently selected from the group consisting of (C₁-C₆)alkyl, (C₂-C₆)alkenyl, (C₁-C₆)perfluoroalkyl, phenyl, hydroxy, —CO₂H, an ester, an amide, (C₁-C₆)alkoxy, (C₁-C₆)thioalkyl and (C₁-C₆)perfluoroalkoxy. However, any of the other suitable substituents known to one skilled in the art can also be used in these embodiments.

It should be noted that any atom with unsatisfied valences in the text, schemes, examples and tables herein is assumed to have the appropriate number of hydrogen atom(s) to satisfy such valences.

It will be understood that the terms “dielectric” and “insulating” are used interchangeably herein. Thus reference to an insulating material or layer is inclusive of a dielectric material or layer and vice versa.

It will be understood that, as used herein, the phrase “microelectronic device” is inclusive of a “micro-optoelectronic device” and an “optoelectronic device”. Thus, reference to microelectronic devices or a microelectronic device assemblies are inclusive of optoelectronic devices and micro-optoelectronic devices as well as assemblies thereof.

It will be understood that the term “redistribution layer (RDL)” refers to an electrical signal routing insulation material which features desirable and reliable properties. The term RDL may also be used interchangeably to describe buffer coating layers, such as for example, a stress relief or buffer layer between the solder ball and fragile low-K structure.

As used herein, the terms “polymer composition,” “copolymer composition,” “terpolymer composition” or “tetrapolymer composition” are used herein interchangeably and are meant to include at least one synthesized polymer, copolymer, terpolymer or tetrapolymer, as well as residues from initiators, solvents or other elements attendant to the synthesis of such polymers, where such residues are understood as not necessarily being covalently incorporated thereto. But some catalysts or initiators may sometimes be covalently bound to a part of the polymeric chain either at the beginning and/or end of the polymeric chain. Such residues and other elements considered as part of the “polymer” or “polymer composition” are typically mixed or co-mingled with the polymer such that they tend to remain therewith when it is transferred between vessels or between solvent or dispersion media. A polymer composition can also include materials added after synthesis of the polymer to provide or modify specific properties of such composition. Such materials include, but are not limited to solvent(s), antioxidant(s), photoinitiator(s), sensitizers and other materials as will be discussed more fully below.

As used herein, the term “modulus” is understood to mean the ratio of stress to strain and unless otherwise indicated, refers to the Young's Modulus or Tensile Modulus measured in the linear elastic region of the stress-strain curve. Modulus values are generally measured in accordance with ASTM method DI708-95. Films having a low modulus are understood to also have low internal stress.

The term “photodefinable” refers to the characteristic of a material or composition of materials, such as a polymer or polymer composition in accordance with embodiments of the present invention, to be formed into, in and of itself, a patterned layer or a structure. In alternate language, a “photodefinable layer” does not require the use of another material layer formed thereover, for example, a photoresist layer, to form the aforementioned patterned layer or structure. It will be further understood that a polymer composition having such a characteristic is generally employed in a pattern forming scheme to form a patterned film/layer or structure. It will be noted that such a scheme incorporates an “imagewise exposure” of the photodefinable material or layer formed therefrom. Such imagewise exposure being taken to mean an exposure to actinic radiation of selected portions of the layer, where non-selected portions are protected from such exposure to actinic radiation.

As used herein, the term “self-imageable compositions” will be understood to mean a material that is photodefinable and can thus provide patterned layers and/or structures after direct image-wise exposure of a film formed thereof followed by development of such images in the film using an appropriate developer.

By the term “derived” is meant that the polymeric repeating units are formed from, for example, condensation of a dianhydride with a diamine. That is, polyimide repeat units are derived from the corresponding dianhydride and diamine. Generally, such condensation reaction first results in a polyamic acid which is further condensed to form a polyimide as described further in detail below. Accordingly, a polyamic acid or a polyimide is generally derived from the condensation of equimolar amounts of at least one dianhydride with one diamine. When a mono-anhydride or a mono-amine is used off-setting the stoichiometry, the resulting polyimide will be end-capped with such excess amount of either the mono-anhydride or the mono-amine employed.

Thus, in accordance with the practice of this invention there is provided a composition comprising:
a) a polyamic acid selected from the group consisting of a polyamic acid of formula (IA), an end capped polyamic acid of formula (IB) and an end capped polyamic acid of formula (IC):

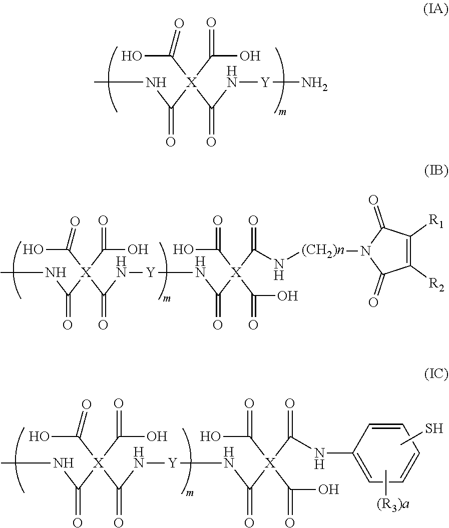

wherein:
m is an integer of at least 50;
n is an integer from 1 to 12, inclusive;
a is an integer from 1 to 4;
X is one or more distinct tetravalent organic group, which is derived from one or more dianhydrides selected from the group consisting of:

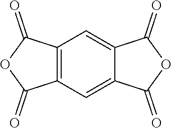 [SMILES: O=c1oc(=O)c2cc3c(=O)oc(=O)c3cc12]

### 1H,3H-benzo[1,2-c:4,5-c′]difuran-1,3,5,7-tetraone (PMDA)

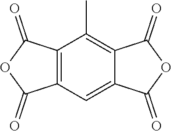 [SMILES: Cc1c2c(=O)oc(=O)c2cc2c(=O)oc(=O)c12]

### 4-methyl-1H,3H-benzo[1,2-c:4,5-c′]difuran-1,3,5,7-tetraone

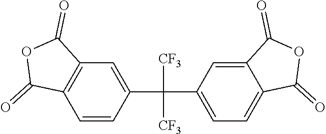 [SMILES: O=C1OC(=O)c2cc(C(c3ccc4c(c3)C(=O)OC4=O)(C(F)(F)F)C(F)(F)F)ccc21]

### 5,5′-(perfluoropropane-2,2-diyl)bis(isobenzofuran-1,3-dione) (6FDA)

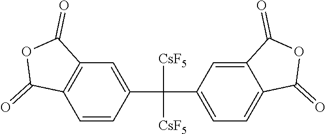

### 5,5′-(perfluoropentane-3,3-diyl)bis(isobenzofuran-1,3-dione)

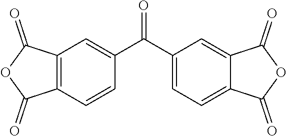 [SMILES: O=C(c1ccc2c(c1)C(=O)OC2=O)c1ccc2c(c1)C(=O)OC2=O]

### 5,5′-carbonylbis(isobenzofuran-1,3-dione) (BTDA)

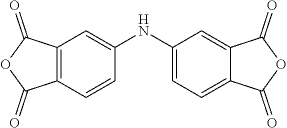 [SMILES: O=C1OC(=O)c2cc(Nc3ccc4c(c3)C(=O)OC4=O)ccc21]

### 5,5′-azanediylbis(isobenzofuran-1,3-dione)

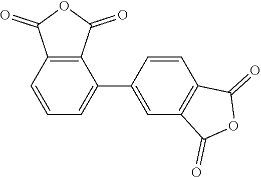 [SMILES: O=C1OC(=O)c2cc(-c3cccc4c3C(=O)OC4=O)ccc21]

### [4,5′-biisobenzofuran]-1,1′,3,3′-tetraone (α-BPDA)

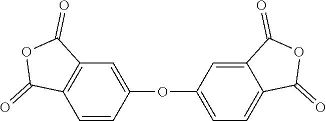 [SMILES: O=C1OC(=O)c2cc(Oc3ccc4c(c3)C(=O)OC4=O)ccc21]

### 5,5′-oxybis(isobenzofuran-1,3-dione) (ODPA)

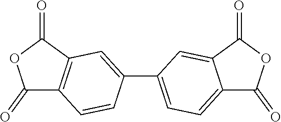 [SMILES: O=C1OC(=O)c2cc(-c3ccc4c(c3)C(=O)OC4=O)ccc21]

### [5,5′-biisobenzofuran]-1,1′,3,3′-tetraone (BPDA); and

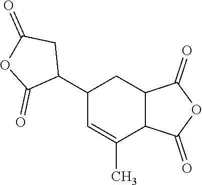 [SMILES: CC1=CC(C2CC(=O)OC2=O)CC2C(=O)OC(=O)C12]

### 5-(2,5-dioxotetrahydrofuran-3-yl)-7-methyl-3a,4,5,7a-tetrahydroisobenzofuran-1,3-dione (D1901)

Y is one or more distinct divalent organic group derived from a diamine; and
R₁ and R₂ are the same or different and each independently of one another selected from the group consisting of hydrogen, linear or branched (C₁-C₁₆)alkyl, perfluoro(C₁-C₁₂)alkyl, linear or branched (C₁-C₁₆)alkenyl, perfluoro(C₁-C₁₂)alkenyl, (C₆-C₁₀)aryl and (C₆-C₁₀)aryl(C₁-C₃)alkyl; or
R₁ and R₂ taken together with the carbon atoms to which they are attached to form a 5 to 7 membered monocyclic ring, a 6 to 12 membered bicyclic ring or a 9 to 14 membered tricyclic ring, said rings optionally containing one or more heteroatoms selected from O, N and S, and said rings optionally substituted with one or more groups selected from the group consisting of methyl, ethyl, linear or branched (C₃-Cs)alkyl, (C₆-C₁₀)aryl, halogen, hydroxy, linear or branched (C₁-C₈)alkoxy and (C₆-C₁₀)aryloxy; and
each of R3 is independently selected from the group consisting of hydrogen, halogen, hydroxy, methyl, ethyl, linear or branched (C₃-C₆)alkyl, trifluoromethyl, pentafluoroethyl, linear or branched perfluoro(C₃-C₆)alkyl, methoxy, ethoxy, linear or branched (C₃-C₆)alkyloxy, (C₂-C₆)acyl, (C₂-C₆)acyloxy, phenyl and phenoxy; and
b) a diazirine compound of formula (II):

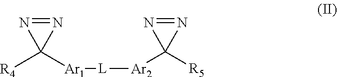

wherein,
L is a bond or a divalent linking or a spacer group selected from the group consisting of: —O—, —O—, —CH₂—, —CHR₆—, —C(R₆)₂—, —C(O)O—R₆—OC(O)—, —C(O)O—R₆—, —R₆—OC(O)—R₆—, —C(O)—R₆—OC(O)—, —C(O)—R₆—, —R₆—C(O)—R₆—, —O—R₆—OC(O)—, —O—R₆—O—, —O—R₆—, —R₆—O—R₆—, —C(O)NH—(CH₂)_{b}—NH(CO)—, where b is an integer from 1 to 15, inclusive, —C(O)NH—(CH₂CH₂O)_{c}(CH₂)ₐ—NR₆(CO)—, where c is an integer from 2 to 6, inclusive and d is an integer from 1 to 6, inclusive, and each occurrence of R₆ may be the same or different which is a divalent group independently selected from the group consisting of methyl, ethyl, linear or branched (C₃-C₁₂)alkyl, (C₃-C₁₂)cycloalkyl, (C₆-C₁₂)aryl, (C₆-C₁₂)aryl(C₁-C₁₂)alkyl, (C₆-C₁₀)heteroaryl, (C₆-C₁₀)heteroaryl(C₁-C₁₂)alkyl, —(CH₂—CH₂—O)ₐ—, where a is an integer from 1 to 10, inclusive;
R₄ and R₅ are the same or different and each independently selected from the group consisting of methyl, ethyl, linear or branched (C₃-C₁₂)alkyl, where portions of hydrogen on alkyl are replaced with fluorine, (C₁-C₁₂)perfluoroalkyl, (C₆-C₁₂)aryl, (C₆-C₁₂)aryl(C₁-C₁₂)alkyl, where portions of hydrogen on alkyl are replaced with fluorine, and (C₆-C₁₂)arylperfluoro(C₁-C₁₂)alkyl; and
Ar₁ and Ar₂ are the same or different and each independently selected from the group consisting of (C₆-C₁₄)arylene, (C₆-C₁₂)heteroarylene, (C₆-C₁₄)aryl(C₁-C₈)alkylene, (C₆-C₁₂)heteroaryl(C₁-C₈)alkylene optionally substituted with a group selected from the group consisting of halogen, —OH, methyl, ethyl, linear or branched (C₃-C₆)alkyl, (C₁-C₄)alkoxy, (C₆-C₁₀)aryl, (C₆-C₁₂)aryloxy, (C₆-C₁₂)aryl(C₁-C₄)alkyl and (C₆-C₁₂)aryl(C₁-C₄)alkyloxy.

As used herein, (C₆-C₁₂)heteroarylene and (C₆-C₁₂)heteroaryl(C₁-C₈)alkylene, includes a variety of imide and imidealkyl groups formed by the condensation of a dicarboxylic acid and an amine. Representative examples of such groups include but not limited to bis(benzimidyl), bis(benzimidyl)methyl, bis(benzimidyl) ether, bis(benzimidyl)methyl ether, and the like.

The polyamic acid of formula (IA) or end capped polyamic acid of formulae (IB) or (IC) of this invention can be synthesized by any of the procedures known to one skilled in the art. Such methods generally include condensation of one or more dianhydrides with one or more diamines essentially in equimolar ratios. Further, suitable amounts of a compound of formula (III) or a compound of formula (IV), respectively, is employed to end cap the resulting polyamic acid to form an end capped polyamic acid of formula (IB) or an end capped polyamic acid of formula (IC). Any of the dianhydrides or diamines in combination with suitable compounds of formulae (III) or (IV) or their equivalent precursor compounds can be employed.

More specifically, the dianhydrides and the diamines that are suitable for forming the polyamic acid of this invention can be represented by the following general formulae (IE) and (IF).

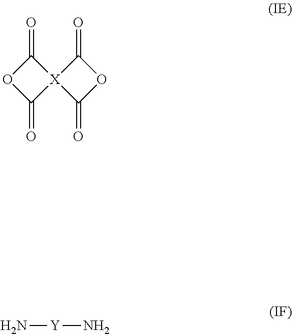

Wherein X and Y are as defined herein. Thus, any of the dianhydrides of tetracarboxylic acids in combination with any of the diamines can be employed to form the polyamic acid and subsequently end capped to form the end capped polyamic acid. Again, as noted, any of the techniques known in the art to make polyamic acid can be employed herein in combination with desirable amounts of the end capping compounds of formulae (III) or (IV), if needed. Now turning specifically to X, any of the suitable tetravalent organic group can be employed herein. Non-limiting examples of such X may be selected from the group consisting of:

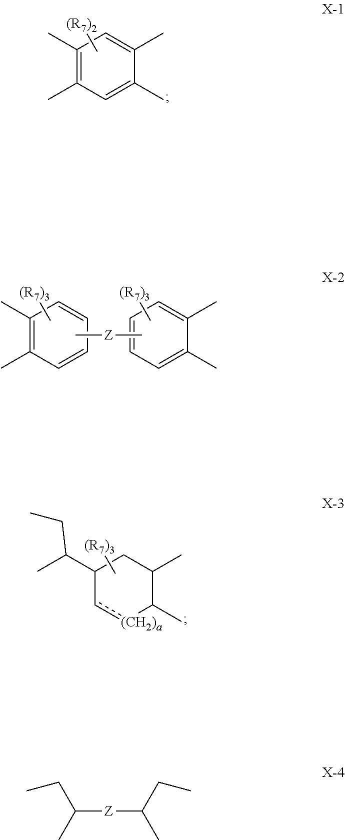

wherein
a is an integer from 0 to 4, inclusive;
[symbol] is a single bond or a double bond;
each of R₇ is independently selected from the group consisting of hydrogen, methyl, ethyl, linear or branched (C₃-C₆)alkyl, trifluoromethyl, pentafluoroethyl, linear or branched perfluoro(C₃-C₆)alkyl, methoxy, ethoxy, linear or branched (C₃-C₆)alkyloxy, (C₂-C₆)acyl, (C₂-C₆)acyloxy, phenyl and phenoxy;
Z is a divalent group selected from the group consisting of:
(CR₈R₉)_{b}, O(CR₈R₉)_{b}, (CR₈R₉)_{b}O, (OCR₈R₉)_{d}, (CR₈R₉O)_{d}, (CR₈R₉)_{b}—O—(CR₈R₉)_{c}, (CR₈R₉)_{b}—O—(SiR₈R₉)_{c}, (CR₈R₉)_{b}—(CO)O—CR₈R₉)_{c}, (CR₈R₉)_{b}—O(CO)—(CR₈R₉)_{c}, (CR₈R₉)_{b}—(CO)—(CR₈R₉)_{c}, (CR₈R₉)_{b}—(CO)NH—(CR₈R₉)_{c}, (CR₈R₉)_{b}—NH(CO)—(CR₈R₉)_{c}, (CR₈R₉)_{b}—NH—(CR₈R₉)_{c}, where b and c are integers which may be the same or different and each independently is 0 to 12, and d is an integer from 1 to 12, inclusive;
R₈ and R₉ are the same or different and each independently selected from the group consisting of hydrogen, methyl, ethyl, linear or branched (C₃-C₆)alkyl, trifluoromethyl, pentafluoroethyl, linear or branched perfluoro(C₃-C₆)alkyl, methoxy, ethoxy, linear or branched (C₃-C₆)alkyloxy, (C₂-C₆)acyl, (C₂-C₆)acyloxy, phenyl and phenoxy.

Even more specifically, suitable dianhydrides may include the following:

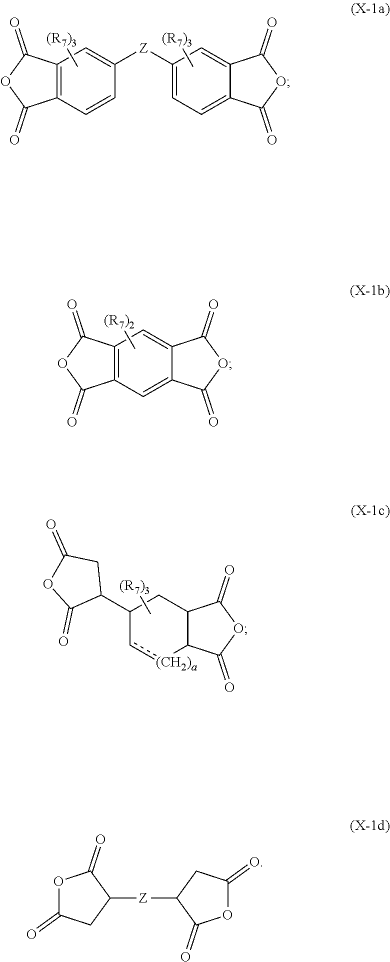

Even more specifically, one or more of the dianhydrides of the following formulae can also be employed herein.

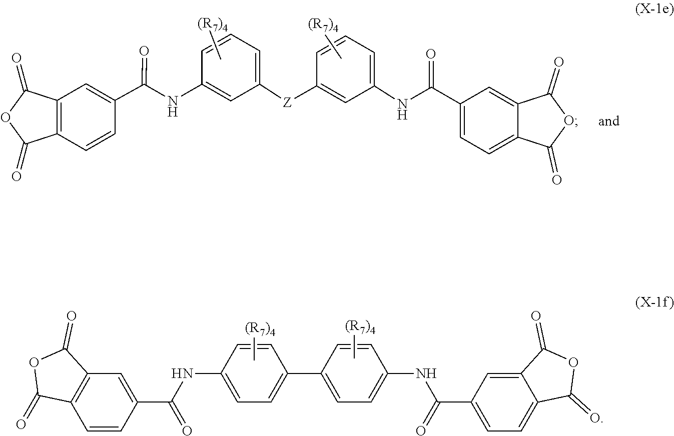

Where a, Z and R₇ are as defined herein.

In some embodiments, the polyamic acid of this invention are formed using the dianhydrides where X is derived from one or more dianhydrides selected from the group consisting of:

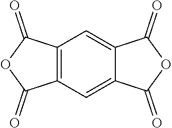 [SMILES: O=c1oc(=O)c2cc3c(=O)oc(=O)c3cc12]

### 1H,3H-benzo[1,2-c:4,5-c′]difuran-1,3,5,7-tetraone (PMDA)

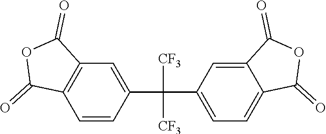 [SMILES: O=C1OC(=O)c2cc(C(c3ccc4c(c3)C(=O)OC4=O)(C(F)(F)F)C(F)(F)F)ccc21]

### 5,5′-(perfluoropropane-2,2-diyl)bis(isobenzofuran-1,3-dione) (6FDA); and

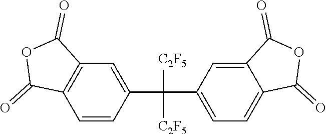

### 5,5′-(perfluoropentane-3,3-diyl)bis(isobenzofuran-1,3-dione)

As noted, again, any of the diamines known in the art can be used to form the polyamic acid of this invention. The diamines can again be broadly classified as aromatic diamines, aliphatic diamines or mixed aliphatic-aromatic diamines which contain a wide variety of bridging groups. A non-limiting generic types of diamines include the following:

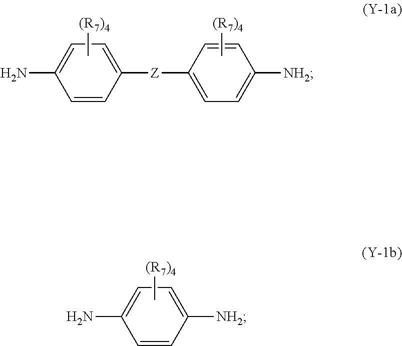

Where a, Z and R₇ are as defined herein.

In some embodiments, the polyamic acid of this invention are formed using the diamines where Y is derived from one or more diamines selected from the group consisting of:

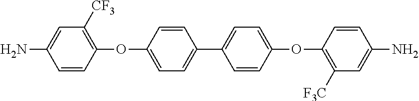 [SMILES: Nc1ccc(Oc2ccc(-c3ccc(Oc4ccc(N)cc4C(F)(F)F)cc3)cc2)c(C(F)(F)F)c1]

### 4,4′-([1,1′-biphenyl]-4,4′-diylbis(oxy))bis(3-(trifluoromethyl)aniline) (6BF)

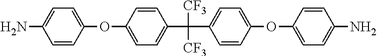 [SMILES: Nc1ccc(Oc2ccc(C(c3ccc(Oc4ccc(N)cc4)cc3)(C(F)(F)F)C(F)(F)F)cc2)cc1]

### 4,4′-(((perfluoropropane-2,2-diyl)bis(4,1-phenylene))bis(oxy))dianiline (HFBAPP)

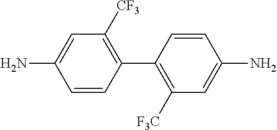 [SMILES: Nc1ccc(-c2ccc(N)cc2C(F)(F)F)c(C(F)(F)F)c1]

### 2,2′-bis(trifluoromethyl)-[1,1′-biphenyl]-4,4′-diamine (PFMB)

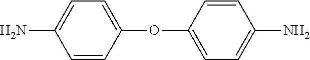 [SMILES: Nc1ccc(Oc2ccc(N)cc2)cc1]

### 4,4′-oxydianiline (4,4′-ODA)

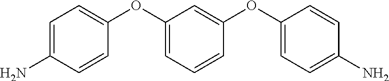 [SMILES: Nc1ccc(Oc2cccc(Oc3ccc(N)cc3)c2)cc1]

### 4,4′-(1,3-phenylenebis(oxy))dianiline (APB)

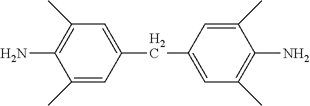 [SMILES: Cc1cc(Cc2cc(C)c(N)c(C)c2)cc(C)c1N]

### 4,4′-methylenebis(2,6-dimethylaniline) (DO3)

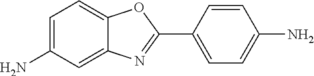 [SMILES: Nc1ccc(-c2nc3cc(N)ccc3o2)cc1]

### 2-(4-aminophenyl)benzo[d]oxazol-5-amine (BZXPh-5)

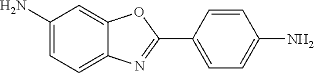 [SMILES: Nc1ccc(-c2nc3ccc(N)cc3o2)cc1]

### 2-(4-aminophenyl)benzo[d]oxazol-6-amine (BZXPh-6)

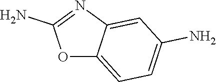 [SMILES: Nc1ccc2oc(N)nc2c1]

### Benzo[d]oxazole-2,5-diamine (BZX-5)

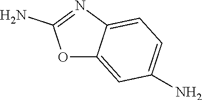 [SMILES: Nc1ccc2nc(N)oc2c1]

### Benzo[d]oxazole-2,6-diamine (BZX-6)

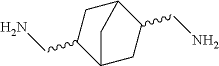 [SMILES: NCC1CC2CC1CC2CN]

### Bicyclo[2.2.1]heptane-2,5-diyldimethanamine (NBDA); a Diamine of Formula (IIIA)

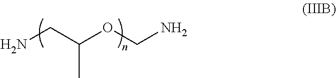

where, n=2 to 6 (JD-230);

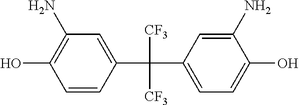 [SMILES: Nc1cc(C(c2ccc(O)c(N)c2)(C(F)(F)F)C(F)(F)F)ccc1O]

### 4,4′-(perfluoropropane-2,2-diyl)bis(2-aminophenol) (BAFA)

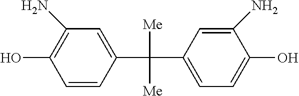 [SMILES: CC(C)(c1ccc(O)c(N)c1)c1ccc(O)c(N)c1]

### 4,4′-(propane-2,2-diyl)bis(2-aminophenol) (DABPA); and and

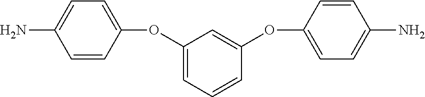 [SMILES: Nc1ccc(Oc2cccc(Oc3ccc(N)cc3)c2)cc1]

### 4,4′-(1,3-phenylenebis(oxy))dianiline (TPE-R)

As noted, the polyamic acid of formulae (IB) and (IC) of this invention are end capped with a respective suitable end capping agent. A suitable end capping agent for forming end capped polyamic acid of formula (IB) is a compound of formula (III):

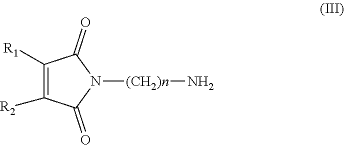

wherein n, R₁ and R₂ are as defined above.

Various compounds of formula (III) are well known in the art and are commercially available or can be made readily following the procedures reported in the art. Again, any of the compounds of formula (III) that will bring about the intended benefit can be employed herein. Non-limiting examples of the compound of formula (III) is selected from the group consisting of:

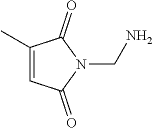

### 1-(aminomethyl)-3-methyl-1H-pyrrole-2,5-dione

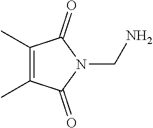

### 1-(aminomethyl)-3,4-dimethyl-1H-pyrrole-2,5-dione

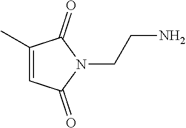

### 1-(2-aminoethyl)-3-methyl-1H-pyrrole-2,5-dione

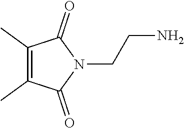

### 1-(2-aminoethyl)-3,4-dimethyl-1H-pyrrole-2,5-dione (DMMIEtNH₂)

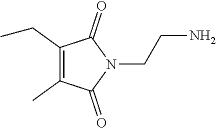

### 1-(2-aminoethyl)-3-ethyl-4-methyl-1H-pyrrole-2,5-dione; and

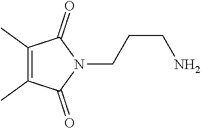

### 1-(3-aminopropyl)-3,4-dimethyl-1H-pyrrole-2,5-dione

Similarly, a suitable end capping agent for forming end capped polyamic acid of formula (IC) is a compound of formula (IV):

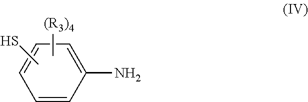

wherein R₃ is as defined above.

Various compounds of formula (IV) are well known in the art and are commercially available or can be made readily following the procedures reported in the art. Again, any of the compounds of formula (IV) that will bring about the intended benefit can be employed herein. Non-limiting examples of the compound of formula (IV) is selected from the group consisting of:

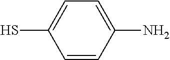

### 4-aminobenzenethiol

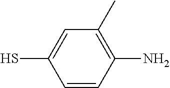

### 4-amino-3-methylbenzenethiol

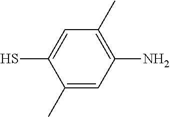

### 4-amino-2,5-dimethylbenzenethiol

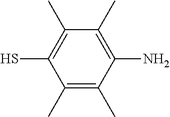

### 4-amino-2,3,5,6-tetramethylbenzenethiol

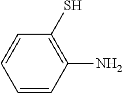

### 2-aminobenzenethiol (2-NH₂PhSH)

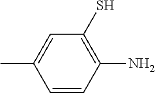

### 2-amino-5-methylbenzenethiol

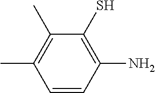

### 6-amino-2.3-dimethylbenzenethiol

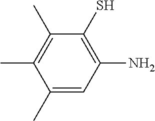

### 6-amino-2,3,4-trimethylbenzenethiol; and

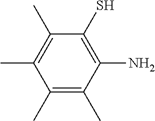

### 2-amino-3,4,5,6-tetramethylbenzenethiol

The polyamic acid of this invention having suitable molecular weight can be tailored based on the intended application by employing appropriate polycondensation methods. Accordingly, in some embodiments the number of repeat units, m, in the resulting polyamic acid is at least about 50; in some other embodiments m is at least 100, 500, 1000, 2000 or higher. In some embodiments m is from 50 to 2000, inclusive. The degree of polycondensation can be measured by determining the molecular weight of the resulting polyamic acid using any of the known methods in the art, such as for example, by gel permeation chromatography (GPC) equipped with suitable detector and calibration standards, such as differential refractive index detector calibrated with narrow-distribution polystyrene standards.

Accordingly, the polyamic acid of this invention generally exhibit a weight average molecular weight (M_{w}) of at least about 20,000. In some other embodiments, the polyamic acid as described herein exhibit a weight average molecular weight (M_{w}) of at least about 50,000. In some other embodiments, the polyamic acid made in accordance of this invention has a M_{w} of at least about 80,000.

In yet another embodiment, the polyamic acid of this invention has a M_{w} of at least about 100,000. In some other embodiments, the polyamic acid of this invention has a M_{w} of at least about 200,000. In some other embodiments, the polyamic acid of this invention has a M_{w} ranging from about 50,000 to 500,000, or higher.

The polyamic acid of this invention generally contains an amic acid repeat unit derived from at least one dianhydride and at least one diamine and if needed end capped with an end capping compound of formula (III) or (IV) as described herein. In some other embodiments, the polyamic acid of this invention contains an amic acid repeat units derived from two or more dianhydrides and one or more diamines as described herein, which is further end capped, if needed, with an end capping compound of formula (III) or (IV) as described herein. All of such permutation and combinations are part of this invention. Generally, equimolar ratios of dianhydrides and diamines are employed to form the polyamic acid or the polyimide. That is, one mole of dianhydride is condensed with one mole of diamine. When two or more dianhydrides or diamines are employed, any of the molar ratios of the respective two or more dianhydrides and/or diamines can be employed so as to tailor the properties of the resulting polyamic acid and depending upon the intended applications, and if needed end capped with an end capping compound of formula (III) or (IV). In any event, the polyamic acid of this invention contains generally equal molar amounts of the total dianhydride and the total diamines when more than one dianhydride or more than one diamine is employed.

That is, a polyamic or the polyimide of this invention is made by employing equimolar amounts of dianhydride and diamine, which includes, if needed, small amounts of an end capping compound of formula (III) or (IV).

Surprisingly, it has now been found that incorporation of small amounts of end capping agents, such as compounds of formulae (III) or (IV), it is now possible to prepare the respective end capped polyamic acids which provide some advantages for the compositions of this invention as it can be appreciated from the specific examples that follow. Generally, any amount of the end capping agent can be used so as to enhance the properties of the resulting end capped polyamic acids. In some embodiments the amount of end capping agent employed is at least two mole percent based on the total moles of dianhydride, diamine and the end capping agent used. In some other embodiments the amount of end capping agent employed is from about two mole percent to about five mole percent based on the total moles of dianhydride, diamine and the end capping agent used. In yet some other embodiments the amount of end capping agent employed can be lower than two mole percent or can be higher than five mole percent based on the total moles of dianhydride, diamine and the end capping agent used.

As noted, the compositions of this invention contain at least one diazirine of formula (II). Various diazirines of formula (II) are well known in the art and are commercially available or can be made readily following the procedures reported in the art. For example, see U.S. Pat. No. 9,938,241 B2, pertinent portions of which are incorporated herein. Again, any of the compounds of formula (II) that will bring about the intended benefit can be employed herein. Exemplary diazirines within the scope of the invention may be enumerated as follows:

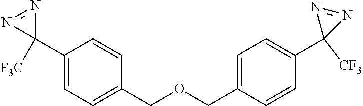 [SMILES: FC(F)(F)C1(c2ccc(COCc3ccc(C4(C(F)(F)F)N=N4)cc3)cc2)N=N1]

### 3,3′-((oxybis(methylene))bis(4,1-phenylene))bis(3-(trifluoromethyl)-3H-diazirine) (PCG-1)

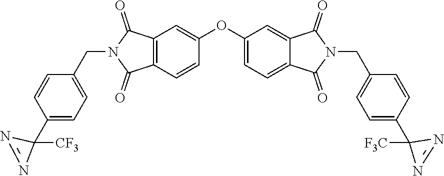 [SMILES: O=C1c2ccc(Oc3ccc4c(c3)C(=O)N(Cc3ccc(C5(C(F)(F)F)N=N5)cc3)C4=O)cc2C(=O)N1Cc1ccc(C2(C(F)(F)F)N=N2)cc1]

### 5,5′-oxybis(2-(4-(3-(trifluoromethyl)-3H-diazirin-3-yl)benzyl)isoindoline-1,3-dione) (PCG-3)

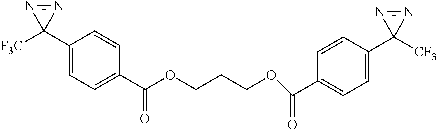 [SMILES: O=C(OCCCOC(=O)c1ccc(C2(C(F)(F)F)N=N2)cc1)c1ccc(C2(C(F)(F)F)N=N2)cc1]

### Propane-1,3-diyl bis(4-(3-(trifluoromethyl)-3H-diazirin-3-yl)benzoate)

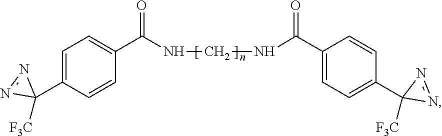

where n is 7 or 12;

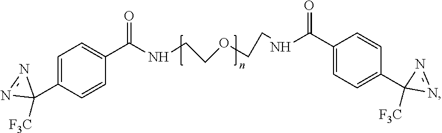

where n is 3 or 5;

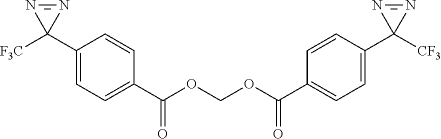 [SMILES: O=C(OCOC(=O)c1ccc(C2(C(F)(F)F)N=N2)cc1)c1ccc(C2(C(F)(F)F)N=N2)cc1]

### Methylene bis(4-(3-(trifluoromethyl)-3H-diazinin-3-yl)benzoate)

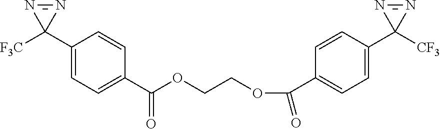 [SMILES: O=C(OCCOC(=O)c1ccc(C2(C(F)(F)F)N=N2)cc1)c1ccc(C2(C(F)(F)F)N=N2)cc1]

### Ethane-1,2-diyl bis(4-(3-(trifluoromethyl)-3H-diazirin-3-yl)benzoate)

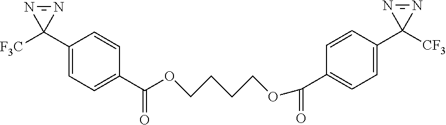 [SMILES: O=C(OCCCCOC(=O)c1ccc(C2(C(F)(F)F)N=N2)cc1)c1ccc(C2(C(F)(F)F)N=N2)cc1]

### Butane-1,4-diyl bis(4-(3-(trifluoromethyl)-3H-diazirin-3-yl benzoate)

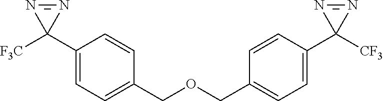 [SMILES: FC(F)(F)C1(c2ccc(COCc3ccc(C4(C(F)(F)F)N=N4)cc3)cc2)N=N1]

### 3,3′-((oxybis(methylene))bis(4,1-phenylene))bis(3-(trifluoromethyl)-3H-diazirine)

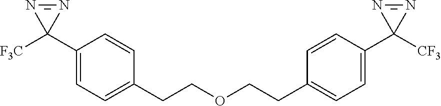 [SMILES: FC(F)(F)C1(c2ccc(CCOCCc3ccc(C4(C(F)(F)F)N=N4)cc3)cc2)N=N1]

### 3,3′-((oxybis(ethane-2,1-diyl))bis(4,1-phenylene))bis(3-(trifluoromethyl)-3H-diazirine)

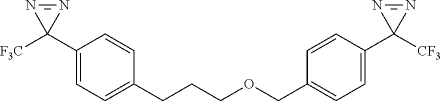 [SMILES: FC(F)(F)C1(c2ccc(CCCOCc3ccc(C4(C(F)(F)F)N=N4)cc3)cc2)N=N1]

### 3-(trifluoromethyl)-3-(4-(3-((4-(3-(trifluoromethyl)-3H-diazirin-3-yl)benzyl)oxy)propyl)phenyl)-3H-diazirine

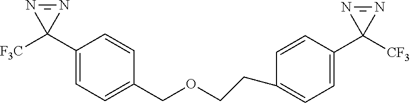 [SMILES: FC(F)(F)C1(c2ccc(CCOCc3ccc(C4(C(F)(F)F)N=N4)cc3)cc2)N=N1]

### 3-(trifluoromethyl)-3-(4-(2-((4-(3-(trifluoromethyl)-3H-diazirin-3-yl)benzyl)oxy)ethyl)phenyl)-3H-diazirine

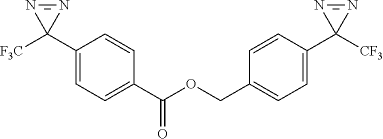 [SMILES: O=C(OCc1ccc(C2(C(F)(F)F)N=N2)cc1)c1ccc(C2(C(F)(F)F)N=N2)cc1]

### 4-(3-(trifluoromethyl)-3H-diazirin-3-yl)benzyl 4-(3-(trifluoromethyl)-3H-diazirin-3-yl)benzoate

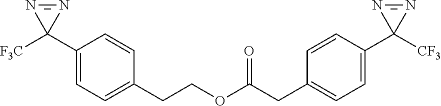 [SMILES: O=C(Cc1ccc(C2(C(F)(F)F)N=N2)cc1)OCCc1ccc(C2(C(F)(F)F)N=N2)cc1]

### 4-(3-(trifluoromethyl)-3H-diazirin-3-yl)phenethyl 2-(4-(3-(trifluoromethyl)-3H-diazirin-3-yl)phenyl)acetate

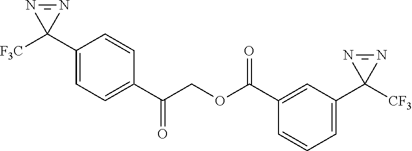 [SMILES: O=C(COC(=O)c1cccc(C2(C(F)(F)F)N=N2)c1)c1ccc(C2(C(F)(F)F)N=N2)cc1]

### 2-oxo-2-(4-(3-(trifluoromethyl)-3H-diazirin-3-yl)phenyl)ethyl 3-(3-(trifluoromethyl)-3H-diazirin-3-yl)benzoate

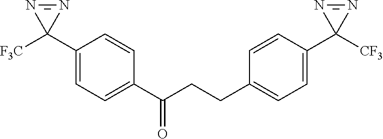 [SMILES: O=C(CCc1ccc(C2(C(F)(F)F)N=N2)cc1)c1ccc(C2(C(F)(F)F)N=N2)cc1]

### 1,3-bis(4-(3-(trifluoromethyl)-3H-diazirin-3-yl)phenyl)propan-1-one

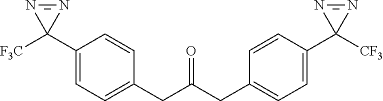 [SMILES: O=C(Cc1ccc(C2(C(F)(F)F)N=N2)cc1)Cc1ccc(C2(C(F)(F)F)N=N2)cc1]

### 1,3-bis(4-(3-(trifluoromethyl)-3H-diazirin-3-yl)phenyl)propan-2-one

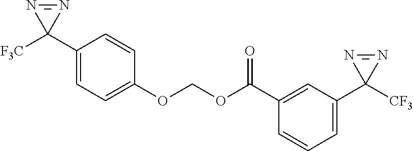 [SMILES: O=C(OCOc1ccc(C2(C(F)(F)F)N=N2)cc1)c1cccc(C2(C(F)(F)F)N=N2)c1]

### (4-(3-(trifluoromethyl)-3H-diazirin-3-yl)phenoxy)methyl 3-(3-(trifluoromethyl)-3H-diazirin-3-yl)benzoate

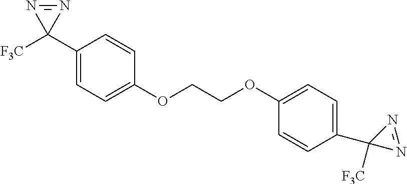 [SMILES: FC(F)(F)C1(c2ccc(OCCOc3ccc(C4(C(F)(F)F)N=N4)cc3)cc2)N=N1]

### 1,2-bis(4-(3-(trifluoromethyl)-3H-diazirin-3-yl)phenoxy)ethane

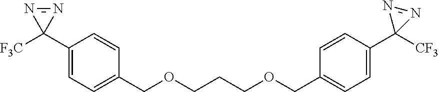 [SMILES: FC(F)(F)C1(c2ccc(COCCCOCc3ccc(C4(C(F)(F)F)N=N4)cc3)cc2)N=N1]

### 1,3-bis((4-(3-(trifluoromethyl)-3H-diazirin-3-yl)benzyl)oxy)propane

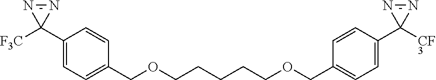 [SMILES: FC(F)(F)C1(c2ccc(COCCCCCOCc3ccc(C4(C(F)(F)F)N=N4)cc3)cc2)N=N1]

### 1,5-bis((4-(3-(trifluoromethyl)-3H-diazirin-3-yl)benzyl)oxy)pentane

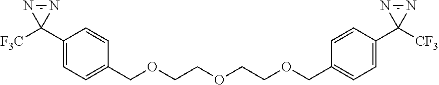 [SMILES: FC(F)(F)C1(c2ccc(COCCOCCOCc3ccc(C4(C(F)(F)F)N=N4)cc3)cc2)N=N1]

### 3,3′-((((oxybis(ethane-2,1-diyl))bis(oxy))bis(methylene))bis(4,1-phenylene))bis(3-(trifluoromethyl)-3H-diazirine)

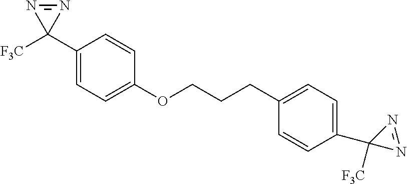 [SMILES: FC(F)(F)C1(c2ccc(CCCOc3ccc(C4(C(F)(F)F)N=N4)cc3)cc2)N=N1]

### 3-(trifluoromethyl)-3-(4-(3-(4-(3-(trifluoromethyl)-3H-diazirin-3-yl)phenoxy)propyl)phenyl)-3H-diazirine

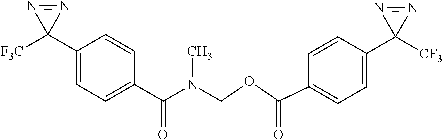 [SMILES: CN(COC(=O)c1ccc(C2(C(F)(F)F)N=N2)cc1)C(=O)c1ccc(C2(C(F)(F)F)N=N2)cc1]

### (N-methyl-4-(3-(trifluoromethyl)-3H-diazirin-3-yl)benzamido)methyl 4-(3-(trifluoromethyl)-3H-diazirin-3-yl)benzoate; and

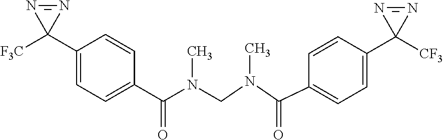 [SMILES: CN(CN(C)C(=O)c1ccc(C2(C(F)(F)F)N=N2)cc1)C(=O)c1ccc(C2(C(F)(F)F)N=N2)cc1]

### N,N′-methylenebis(N-methyl-4-(3-(trifluoromethyl)-3H-diazirin-3-yl)benzamide)

Advantageously, it has now been found that diazirines having the bis-imide groups, such as bis-imide ether linking group, as described specifically herein, provides synergistic results in the composition of this invention. For example, a diazirine of the formula as used in the composition of this invention is found to provide beneficial effects:

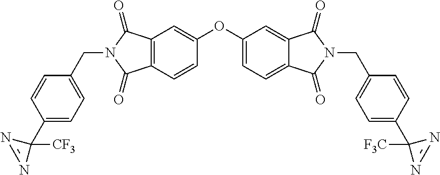 [SMILES: O=C1c2ccc(Oc3ccc4c(c3)C(=O)N(Cc3ccc(C5(C(F)(F)F)N=N5)cc3)C4=O)cc2C(=O)N1Cc1ccc(C2(C(F)(F)F)N=N2)cc1]

5,5′-oxybis(2-(4-(3-(trifluoromethyl)-3H-diazirin-3-yl)benzyl)isoindoline-1,3-dione) (PCG-3). PCG-3 can be prepared using any of the similar procedures as reported in the prior art methods.

Accordingly, a variety of bis-imide diazirines of formula (IIA) can be used in the composition of this invention. A representative preparation of diazirines of formula (IIA) is shown in Scheme I.

In Scheme I, Step 1, PCG-precursor, (4-(3-(trifluoromethyl)-3H-diazirin-3-yl)phenyl)methanamine or a salt thereof, such as for example with hydrochloric acid, is reacted with a dianhydride to form bis-diazirine amic acid. Any of the dianhydrides as described herein can be employed in this step, where X is as defined herein. For example, by employing ODPA, it is now possible to form PCG-2, an amic acid precursor, 4,4′-oxybis(2-((4-(3-(trifluoromethyl)-3H-diazirin-3-yl)benzyl)carbamoyl)benzoic acid). This reaction can be carried out using any of the known methods in the art. For example, a solution of PCG-precursor is reacted with a dianhydride, such as ODPA in the presence of a suitable base to form bis-diazirine amic acid, such as PCG-2. Suitable solvents include ether solvents such as tetrahydrofuran (THF), ketone solvents such as acetone or halogenated solvents such as dichloromethane and the like or mixtures in combination thereof. Suitable base include trialkyl amines, such as for example Hunig's base. Various other bases that can be used may include alkaline metal or alkaline earth metal carbonate or bicarbonate, such as sodium carbonate, potassium carbonate or ammonium carbonate, and the like.

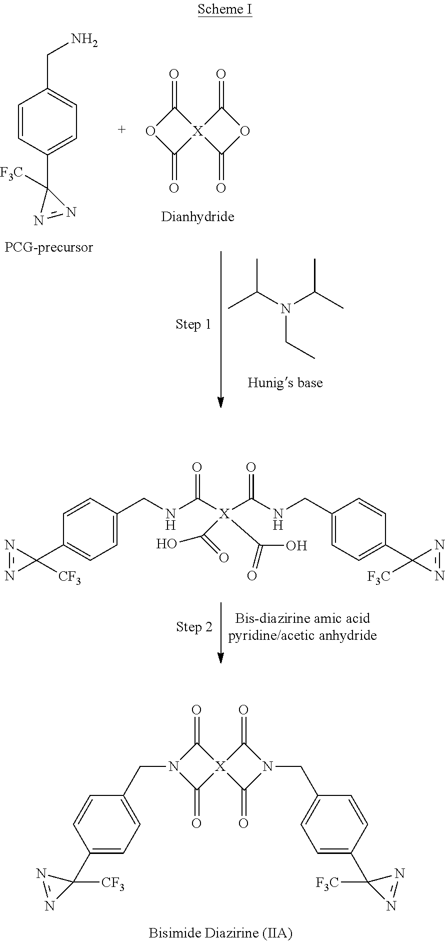

The reaction can be carried out at ambient, sub-ambient or super-ambient temperature conditions. Generally, the reaction temperature can range from about 20° C. to 40° C., but higher temperatures can be employed depending upon the type of dianhydrides used to form the bis-diazirine amic acid, such as PCG-2. Again, any of the dianhydrides as described herein are suitable for forming the corresponding bis-diazirine amic acid.

In Scheme I, Step 2, bis-diazirine amic acid is reacted with a suitable condensation agent, such as for example an equimolar mixture of pyridine and acetic anhydride, to form bis-imide diazirine, such as, PCG-3, 5,5′-oxybis(2-(4-(3-(trifluoromethyl)-3H-diazirin-3-yl)benzyl)isoindoline-1,3-dione). This reaction can be carried out using any of the known methods in the art. For example, a solution of bis-diazirine amic acid is reacted with a mixture of pyridine and acetic anhydride at a suitable temperature conditions to form the corresponding bis-imide diazirine, such as, PCG-3.

Suitable solvents include dimethyl sulfoxide (DMSO), N-methyl-2-pyrrolidone (NMP), γ-butyrolactone (GBL), N,N-dimethylacetamide (DMAc), propylene glycol monomethyl ether acetate (PGMEA), and the like or mixtures in combination thereof. The reaction can be carried out at ambient, sub-ambient or super-ambient temperature conditions. Generally, the reaction temperature can range from about 20° C. to 40° C., but higher temperatures can be employed to form the bis-diazirine imide, such as PCG-3.

Any amount of diazirine compound of formula (II) can be employed in the composition of this invention which brings about the intended benefit. In some embodiments the amount of diazirine compound of formula (II) employed in the composition is at least 15 parts per 100 parts of amic acid of formulae (IA), (IB) or (IC) used. That is, at least 15 parts per hundred parts resin (pphr) of diazirine compound of formula (II) is used per 100 parts of amic acid of formulae (IA), (IB) or (IC). In some other embodiments the amount of diazirine compound of formula (II) employed in the composition is from about 20 pphr to about 40 pphr, in some other embodiments it is from 25 pphr to about 35 pphr, and so on. However, it should be noted that lower than 15 pphr or higher than 40 pphr of diazirine compound of formula (II) can also be used depending upon the type of amic acid used as well as the intended application.

Surprisingly, it has now been found that the composition of this invention need not contain any of the other known crosslinkers, such as for example acrylate crosslinkers. Even more importantly the composition of this invention is unique as it does not require any of the known photoradical generators and/or photoacid generators. Even in the absence of such additives, the composition of this invention exhibits both photoimaging ability as well as it can be thermally cured to form a variety of objects, such as for example films, sheets having utility in a variety of electronic and opto-electronic applications.

Non-limiting examples of compositions in accordance of this invention may be enumerated as follows:

A polyamic acid formed from 1H,3H-benzo[1,2-c:4,5-c′]difuran-1,3,5,7-tetraone (PMDA), 4,4′-(1,3-phenylenebis(oxy))dianiline (APB), a diamine of formula (ID) (JD230) and 3,3′-((oxybis(methylene))bis(4,1-phenylene))bis(3-(trifluoromethyl)-3H-diazirine) (PCG-1).

A polyamic acid formed from 5,5′-(perfluoropropane-2,2-diyl)bis(isobenzofuran-1,3-dione) (6FDA), 1H,3H-benzo[1,2-c:4,5-c′]difuran-1,3,5,7-tetraone (PMDA), 4,4′-([1,1′-biphenyl]-4,4′-diylbis(oxy))bis(3-(trifluoromethyl)aniline) (6BF), 2,2′-bis(trifluoromethyl)-[1,1′-biphenyl]-4,4′-diamine (PFMB) and 3,3′-((oxybis(methylene))bis(4,1-phenylene))bis(3-(trifluoromethyl)-3H-diazirine) (PCG-1).

A polyamic acid formed from 5,5′-(perfluoropropane-2,2-diyl)bis(isobenzofuran-1,3-dione) (6FDA), 1H,3H-benzo[1,2-c:4,5-c′]difuran-1,3,5,7-tetraone (PMDA), 4,4′-([1,1′-biphenyl]-4,4′-diylbis(oxy))bis(3-(trifluoromethyl)aniline) (6BF), 2,2′-bis(trifluoromethyl)-[1,1′-biphenyl]-4,4′-diamine (PFMB), 1-(2-aminoethyl)-3,4-dimethyl-1H-pyrrole-2,5-dione (DMMIEtNH₂) and 3,3′-((oxybis(methylene))bis(4,1-phenylene))bis(3-(trifluoromethyl)-3H-diazirine) (PCG-1).

A polyamic acid formed from 5,5′-(perfluoropropane-2,2-diyl)bis(isobenzofuran-1,3-dione) (6FDA), 1H,3H-benzo[1,2-c:4,5-c′]difuran-1,3,5,7-tetraone (PMDA), 4,4′-([1,1′-biphenyl]-4,4′-diylbis(oxy))bis(3-(trifluoromethyl)aniline) (6BF), 2,2′-bis(trifluoromethyl)-[1,1′-biphenyl]-4,4′-diamine (PFMB), 1-(2-aminoethyl)-3,4-dimethyl-1H-pyrrole-2,5-dione (DMMIEtNH₂) and 5,5′-oxybis(2-(4-(3-(trifluoromethyl)-3H-diazirin-3-yl)benzyl)isoindoline-1,3-dione) (PCG-3).

A polyamic acid formed from 5,5′-(perfluoropropane-2,2-diyl)bis(isobenzofuran-1,3-dione) (6FDA), 1H,3H-benzo[1,2-c:4,5-c′]difuran-1,3,5,7-tetraone (PMDA), 4,4′-([1,1′-biphenyl]-4,4′-diylbis(oxy))bis(3-(trifluoromethyl)aniline) (6BF), 2,2′-bis(trifluoromethyl)-[1,1′-biphenyl]-4,4′-diamine (PFMB), 2-aminobenzenethiol (2-NH₂PhSH) and 3,3′-((oxybis(methylene))bis(4, 1-phenylene))bis(3-(trifluoromethyl)-3H-diazirine) (PCG-1).

A polyamic acid formed from 5,5′-(perfluoropropane-2,2-diyl)bis(isobenzofuran-1,3-dione) (6FDA), 1H,3H-benzo[1,2-c:4,5-c′]difuran-1,3,5,7-tetraone (PMDA), 4,4′-([1,1′-biphenyl]-4,4′-diylbis(oxy))bis(3-(trifluoromethyl)aniline) (6BF), 4,4′-methylenebis(2,6-dimethylaniline) (DO3), 1-(2-aminoethyl)-3,4-dimethyl-1H-pyrrole-2,5-dione (DMMIEtNH₂) and 3,3′-((oxybis(methylene))bis(4, 1-phenylene))bis(3-(trifluoromethyl)-3H-diazirine) (PCG-1).

A polyamic acid formed from 5,5′-(perfluoropropane-2,2-diyl)bis(isobenzofuran-1,3-dione) (6FDA), 1H,3H-benzo[1,2-c:4,5-c′]difuran-1,3,5,7-tetraone (PMDA), 4,4′-([1,1′-biphenyl]-4,4′-diylbis(oxy))bis(3-(trifluoromethyl)aniline) (6BF), 4,4′-methylenebis(2,6-dimethylaniline) (DO3), 1-(2-aminoethyl)-3,4-dimethyl-1H-pyrrole-2,5-dione (DMMIEtNH₂) and 5,5′-oxybis(2-(4-(3-(trifluoromethyl)-3H-diazirin-3-yl)benzyl)isoindoline-1,3-dione) (PCG-3).

Advantageously, all of the polyamic acids used in the composition of this invention are soluble in an organic solvent. Exemplary organic solvents, without any limitation, that can be employed to dissolve the polyamic acid of this invention are selected from the group consisting of N-methyl-2-pyrrolidone (NMP), γ-butyrolactone (GBL), N,N-dimethylacetamide (DMAc), propylene glycol monomethyl ether acetate (PGMEA), dimethyl sulfoxide (DMSO), cyclopentanone, cyclohexanone, 2-butanone and 2-heptanone and mixtures in any combination thereof. As noted, any of the aforementioned solvents can be used alone or in combination with one or more solvents.

In some embodiments the composition of this invention may further contain one or more epoxy crosslinking agents selected from the group consisting of:

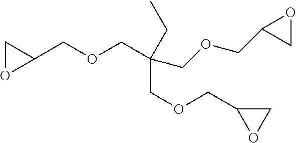 [SMILES: CCC(COCC1CO1)(COCC1CO1)COCC1CO1]

### 2,2′-(((2-ethyl-2-((oxiran-2-ylmethoxy)methyl)propane-1,3-diyl)bis(oxy))bis(methylene))bis(oxirane) (TMPTGE)

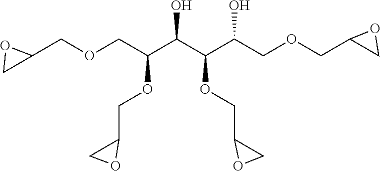 [SMILES: O[C@@H]([C@H](OCC1CO1)[C@H](O)COCC1CO1)[C@H](COCC1CO1)OCC1CO1]

### (2R,3R,4R,5S)-1,3,5,6-tetrakis(oxiran-2-ylmethoxy)hexane-2,4-diol (also known as tetrakis-O—(oxiranylmethyl)-D-glucitol) (Denacol EX-614 From Nagase)

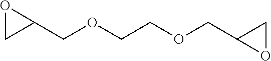 [SMILES: C(COCC1CO1)OCC1CO1]

### 1,2-bis(oxiran-2-ylmethoxy)ethane

a compound of formula (V):

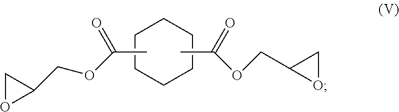

and a compound of formula (VI):

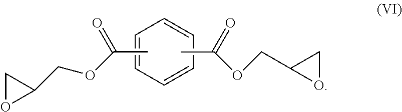

Various other types of epoxy crosslinking agents that can be employed in the compositions of this invention include:

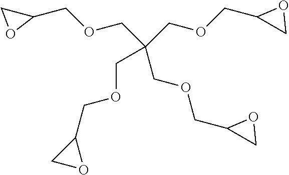 [SMILES: C(OCC(COCC1CO1)(COCC1CO1)COCC1CO1)C1CO1]

### 2,2′-(((2,2-bis((oxiran-2-ylmethoxy)methyl)propane-1,3-diyl)bis(oxy))bis(methylene))bis(oxirane), commercially available as PEGE from Alfa Aesar

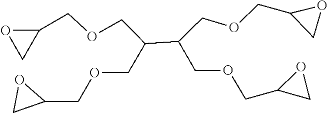 [SMILES: C(OCC(COCC1CO1)C(COCC1CO1)COCC1CO1)C1CO1]

### 2,2′-(((2-(1,3-bis(oxiran-2-ylmethoxy)propan-2-yl)propane-1,3-diyl)bis(oxy))bis(methylene))bis(oxirane)

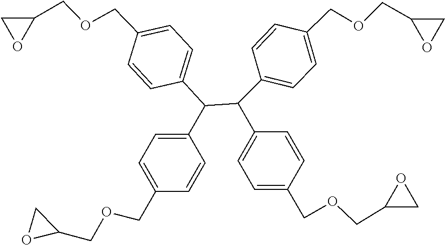 [SMILES: c1cc(C(c2ccc(COCC3CO3)cc2)C(c2ccc(COCC3CO3)cc2)c2ccc(COCC3CO3)cc2)ccc1COCC1CO1]

### 1,1,2,2-tetrakis(4-((oxiran-2-ylmethoxy)methyl)phenyl)ethane

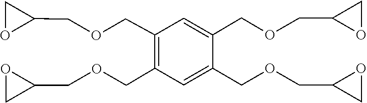 [SMILES: c1c(COCC2CO2)c(COCC2CO2)cc(COCC2CO2)c1COCC1CO1]

### 1,2,4,5-tetrakis((oxiran-2-ylmethoxy)methyl)benzene; and

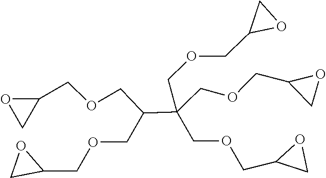 [SMILES: C(OCC(COCC1CO1)C(COCC1CO1)(COCC1CO1)COCC1CO1)C1CO1]

### 2,2′-(((2-(1,3-bis(oxiran-2-ylmethoxy)propan-2-yl)-2-((oxiran-2-ylmethoxy)methyl)propane-1,3-diyl)bis(oxy))bis(methylene))bis(oxirane)

Other suitable crosslinking agents including the following:

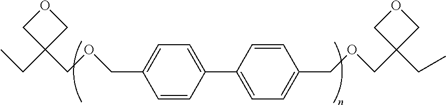

Where n=1 to 3 (OXBP), e.g., when n=1 4,4′-bis(((3-ethyloxetan-3-yl)methoxy)methyl)-1,1′-biphenyl;

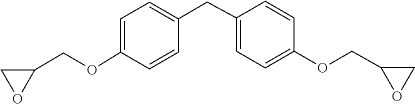 [SMILES: c1cc(OCC2CO2)ccc1Cc1ccc(OCC2CO2)cc1]

### Bis(4-(oxiran-2-ylmethoxy)phenyl)methane

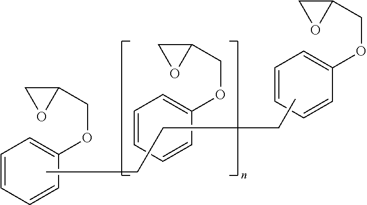

### Phenol-Formaldehyde Polymer Glycidyl Ether, Where n=1 to 10 (EPON 862)

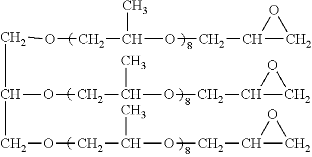 [SMILES: CC(COCC(COCC(C)OCC1CO1)OCC(C)OCC1CO1)OCC1CO1]

### Triglycidyl Ether of Poly(Oxypropylene)Epoxide Ether of Glycerol, Commercially Available as Heloxy 84 or GE-36 from Momentive Specialty Chemicals Inc

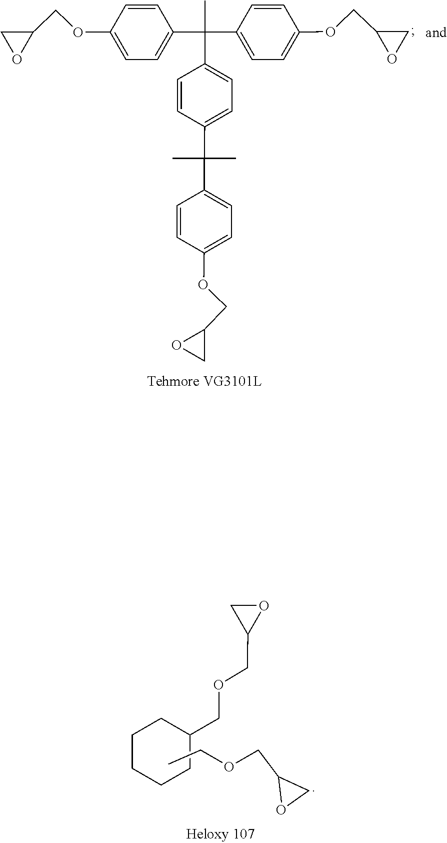

It should further be noted that the composition of this invention can also contain various other known epoxy-type groups containing compounds, including a glycidyl group, an epoxycyclohexyl group, an oxetane group; an oxazoline group such as 2-oxazoline-2-yl group, and the like. Other exemplary cross-linking or crosslinkable materials that can be used in the composition of the present invention include, among others, bisphenol A epoxy resin, bisphenol F epoxy resin, silicone containing epoxy resins or the like, propylene glycol diglycidyl ether, polypropylene glycol diglycidyl ether, glycidyloxypropyltrimethoxysilane, polymethyl (glycidyloxypropyl)cyclohexane or the like; polymers containing oxazoline rings such as 2-methyl-2-oxazoline, 2-ethyl-2-oxazoline, 1,3-bis(2-oxazoline-2-yl)benzene, 1,4-bis(2-oxazoline-2-yl)benzene, 2,2′-bis (2-oxazoline), 2,6-bis(4-isopropyl-2-oxazoline-2-yl)pyridine, 2,6-bis(4-phenyl-2-oxazoline-2-yl)pyridine, 2,2′-isopropylidenebis (4-phenyl-2-oxazoline), (S,S)-(−)-2,2′-isopropylidenebis (4-tert-butyl-2-oxazoline), poly(2-propenyl-2-oxazoline) or the like; or mixtures thereof. It has been found that, in general, such materials are effective at loadings from 5 pphr polyamic acid to 30 pphr polyamic acid. However it should be understood that loadings higher or lower may also prove effective as their efficacy is dependent, at least in part, on the nature of the polyamic acid employed and its mole percent of repeat units encompassing crosslinkable pendent groups.

Accordingly, there is further provided a composition encompassing all of the polyamic acids of formula (IA) or an end capped polyamic acids of formulae (IB) or (IC) as described hereinabove and hereafter, including the specific polyamic acids enumerated above and specifically exemplified below in combination with a diazirine of formula (II) and one or more epoxy crosslinking agent as described above.

The photosensitive composition of this invention may further encompass one or more compounds or additives having utility as, among other things, adhesion promoter, a surface leveling agent, antioxidants, a synergist, silane coupling agents, phenolic resins, flame retardants, plasticizers, curing accelerators, and the like. Examples of surface leveling agents include a variety of non-ionic, amphoteric and anionic surfactants available in the art, which provide, among other things, wetting, spreading and levelling properties. Exemplary surface leveling agents include without any limitation, non-ionic polymeric fluorochemical surfactant, such as for example, FC-4432 available from 3M Advanced Materials Division, a short chain perfluoro-based ethoxylated nonionic fluorosurfactant, such as for example, Chemguard S-550, CAPSTONE fluorosurfactants available as both nonionic and amphoteric forms from DuPont, PolyFox fluorosurfactants from OMNOVA Solutions, and the like. In addition, any of the known conventional surfactants may be used in combination with the above noted surfactants, such known non-ionic surfactants include for example, perfluoroalkyl polyoxyethylene ethanols, fluorinated alkyl esters, perfluoroalkylamine oxides and fluorinated organosiloxane compounds. Various other such commercially available surfactants include Florade FC-4430 from Sumitomo 3M Ltd., Surflon S-141 and S-145 from Asahi Glass Co., Ltd., Unidyne DS-401, DS-4031 and DS-451 from Daikin Industries Ltd., Megaface F-8151 from Dainippon Ink & Chemicals, Inc., and X-70-093 from Shin-Etsu Chemical Co., Ltd.

Non-limiting examples of such other compounds or additives are selected from the group consisting of the following, commercially available materials are indicated by such commercial names.

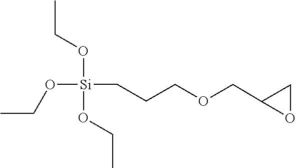 [SMILES: CCO[Si](CCCOCC1CO1)(OCC)OCC]

### Triethoxy(3-(oxiran-2-ylmethoxy)propyl)silane, Also Known as 3-glycidoxypropyl triethoxysilane (3-GTS or (KBE-403 From Shin-Etsu Chemical Co., Ltd.))

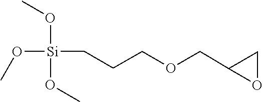

### Trimethoxy(3-(oxiran-2-ylmethoxy)propyl)silane, Also Commonly Known as 3-glycidoxypropyl trimethoxysilane (KBM-403E From Shin-Etsu Chemical Co., Ltd.)); C₆H₅(CH₃O)₃Si phenyltrimethoxysilane C₆H₅(C₂H₅O)₃Si phenyltriethoxysilane (KBE-103 Commercially Available From Gelest or Shin-Etsu Chemical Co., Ltd.)

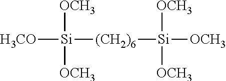

### 3,3,10,10-tetramethoxy-2,11-dioxa-3,10-disiladodecane (SIB-1832 from Gelest)

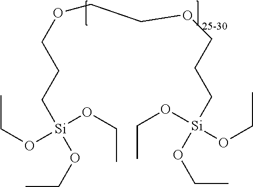

### N,N′-bis[(3-triethoxysilylpropyl)aminocarbonyl]polyethylene oxide (SIB-1824.84 From Gelest)

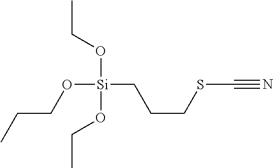 [SMILES: CCCO[Si](CCCSC#N)(OCC)OCC]

### Diethoxy(propoxy)(3-thiocyanatopropyl)silane (SIT 7908.0 From Gelest)

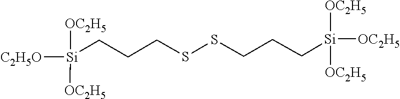

### 4,4,13,13-tetraethoxy-3,14-dioxa-8,9-dithia-4, 13-disilahexadecane

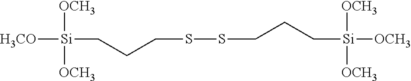 [SMILES: CO[Si](CCCSSCCC[Si](OC)(OC)OC)(OC)OC]

### 3,3,12,12-tetramethoxy-2,13-dioxa-7,8-dithia-3,12-disilatetradecane (Si-75 or Si-266 From Evonik)

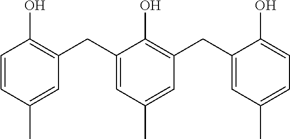 [SMILES: Cc1ccc(O)c(Cc2cc(C)cc(Cc3cc(C)ccc3O)c2O)c1]

### 2,2′-((2-hydroxy-5-methyl-1,3-phenylene)bis(methylene))bis(4-methylphenol) (Antioxidant AO-80 From TCI Japan)

In general among other things, various compounds and additives as enumerated herein improve overall performance of the photosensitive composition of this invention thus providing well defined photo-patterned structures having a variety of utilities, including but not limited to chip-stack applications, redistribution layers (RDL) and for forming CMOS image sensor dam structures. Advantageously, it has also been found that certain of the additives as described herein may feature more than one function. For example, some of the additives as enumerated hereinabove may not only exhibit certain photosensitization activity during exposure to radiation but may also facilitate as a cross linking agent as further described above. Therefore, additives as used herein do not limit the activity of such compounds to only one of such property but may also facilitate other functions of the photosensitive compositions of this invention.

The photosensitive composition embodiments, in accordance with the present invention, are first applied to a desired substrate to form a film. Such a substrate includes any appropriate substrate as is, or may be used for electrical, electronic or optoelectronic devices, for example, a semiconductor substrate, a ceramic substrate, a glass substrate. With regard to said application, any appropriate coating method can be employed, for example, spin coating, spraying, doctor blading, meniscus coating, ink jet coating and slot coating.

Next, the coated substrate is heated to facilitate the removal of residual casting solvent, for example to a temperature from 70° C. to 130° C. for from 1 to 30 minutes, although other appropriate temperatures and times can be used. After the heating, the film is generally imagewise exposed to an appropriate wavelength of actinic radiation, wavelength is generally selected based on the type of diazirine employed and/or optionally a photosensitizer incorporated into the composition. However, generally such appropriate wavelength is from 200 to 700 nm. It will be understood that the phrase “imagewise exposure” means exposing through a masking element to provide for a resulting pattern of exposed and unexposed portion of the film, as further illustrated by specific examples hereinbelow.

After an imagewise exposure of the film formed from photosensitive composition in accordance with the present invention, a development process is employed. For the negative tone compositions as contemplated by the present invention, such development process removes only the unexposed portions of the film thus leaving a negative image of the masking layer in the film. A post exposure bake (PEB) is generally not needed but can be employed prior to the aforementioned development process, generally at a moderately lower temperature from 90° C. to 130° C. for from 1 to 10 minutes, although other appropriate temperatures and times can be used.

Suitable developers can include organic solvents such as propylene glycol methyl ether acetate (PGMEA), 2-heptanone, cyclohexanone, NMP, GBL, cyclopentanone, butyl acetate, and mixtures in any combination thereof, among others.

Thus some composition embodiments of the present invention provide self-imageable films that after imagewise exposure, the resulting image is developed using an organic solvent. After the image is developed, the substrate is rinsed to remove excess developer solution, typical rinse agents are water or appropriate alcohols and mixtures thereof. The excess developer can also be removed by blowing a stream of nitrogen on to the substrate. Other methods of removing excess developer include spinning the developed wafer at high spin speeds of about 1000-3000 rpm for 10-30 sec followed by applying a stream of nitrogen.

After the aforementioned rinsing, the substrate is dried and the imaged film finally cured. That is to say, the image is fixed. Where the remaining layer has already been exposed during the imagewise exposure, image fixing is generally accomplished by causing further reaction within the remaining portions of the film. Such reaction is generally a cross-linking reaction that can be initiated by heating and/or non-imagewise or blanket exposure of the remaining material. Such exposure and heating can be in separate steps or combined as is found appropriate for the specific use of the imaged film. The blanket exposure is generally performed using the same energy source as employed in the imagewise exposure or a higher energy source and may be for a longer period of time although any other appropriate energy source can be employed. The heating is generally carried out at a desirable temperature, for example, from above 150° C. for a time of from 40 min to one or more hours. Where the remaining layer has been exposed during the imagewise exposure, image fixing is generally accomplished by a heating step to be tailored to complete any reaction initiated by the exposure. However an additional blanket exposure and heating, as discussed above, can also be employed. It should be realized, however, that the choice of a final cure process is also a function of the type of device being formed; thus a final fixing of the image may not be a final cure where the remaining layer is to be used as an adhesive layer or structure.

The devices are produced by using embodiments of the composition of the present invention to form layers which are characterized as having high heat resistance, an appropriate water absorption rate, high transparency, and low permittivity. In addition, such layers generally have an advantageous thermo-mechanical properties. Most notably, improved tensile strength, improved elongation to break (ETB) and exhibit higher glass transition temperatures (T_{g}) when compared with conventional materials. For example, the tensile strength of the fully cured composition layer may be higher than 50 MPa and may be in the range of from about 70 MPa to 200 MPa or higher. The ETB of the cured composition layers can be higher than 30 percent and may range from about 50 percent to 100 percent or higher. The T_{g} of the cured composition layer may be higher than 150° C. and can range from about 150° C. to 200° C. or higher. The coefficient of thermal expansion (CTE) may be lower than 100 ppm/K and can range from 30 ppm/K to 100 ppm/K. Young's Modulus of the cured films can be higher than 2 GPa and can range from 2.5 GPa to 4 GPa. It should further be noted that the layers formed in this fashion from the composition of this invention also exhibit unusually high thermal decomposition temperature. Accordingly, the 5 percent weight loss temperature (T_{d5}) of the cured polymeric layers is generally higher than 300° C. and can range from 300° C. to 420° C. or higher, thus offering hitherto unattainable properties.

As previously mentioned, exemplary applications for embodiments of the photosensitive compositions in accordance with the present invention include die attach adhesive, wafer bonding adhesive, insulation films (interlayer dielectric layers), protecting films (passivation layers), mechanical buffer films (stress buffer layers) or flattening films for a variety of semiconductor devices, and printed wiring boards. Specific applications of such embodiments encompass a die-attach adhesive to form a single or multilayer semiconductor device, dielectric film which is formed on a semiconductor device; a buffer coat film which is formed on the passivation film; an interlayer insulation film which is formed over a circuit formed on a semiconductor device.

Accordingly, some embodiments in accordance with the present invention therefore provide a negative tone photosensitive polymer composition which exhibits enhanced characteristics with respect to one or more of mechanical properties (such as high tensile strength, elongation to break) and at least equivalent or better chemical resistance, as compared to alternate materials. In addition, such embodiments provide generally excellent electrical insulation, adhesion to the substrate, and the like. Thus semiconductor devices, device packages, and display devices are provided that incorporate embodiments in accordance with the present invention.

Advantageously, the photosensitive compositions of this invention can also be used to form adhesive layers for bonding the semiconductor chips to each other, such as in chip-stack applications. For example, a bonding layer used for such a purpose is composed of a cured product of the photosensitive adhesive composition of the present invention. It should be noted that although the adhesive layer is a single-layer structure, it can not only exhibit sufficient adhesiveness to the substrate but also it is expected to be free of significant stress resulting due to the curing step. Accordingly, it is now possible to avoid undesirably thick layer of film encompassing the chip as a laminate. Therefore, it should be noted that the laminates formed in accordance with the present invention are expected to be reliable in that the relaxation of stress concentration between layers caused by thermal expansion difference or the like can be obtained. As a result, the semiconductor device having low height and high reliability can be obtained. That is, devices with low aspect ratio and low thickness can be obtained. Such semiconductor device becomes particularly advantageous to electronic equipment, which has very small internal volume and is in use while carrying as a mobile device, for example. Even more advantageously, by practice of this invention it is now possible to form a variety of electronic devices featuring hitherto unachievable level of miniaturization, thinning and light-weight, and the function of the semiconductor device is not easily damaged even if such devices are subject to rugged operations such as swinging or dropping.

Thus, it is now envisioned that the photosensitive adhesive composition of the present invention may exhibit an indentation modulus at room temperature after curing which is relatively comparable to the indentation modulus of the uncured sample and not causing significant stress concentration between the semiconductor chips but contributing to forming of the adhesive layer with sufficient adhesiveness. Further, since the indentation modulus in a state before cured is within the predetermined range of indentation modulus after cured, and then, for example, it is not so possible that the photosensitive adhesive composition before cured is significantly deformed or flowed out, it is possible to increase the accuracy of alignment in laminating the semiconductor chips. Furthermore, since the change in indentation modulus before and after curing is relatively small, the shrinkage associated with photosensitivity can be reduced and then the stress at the interface between the semiconductor chips caused by shrinkage on curing can be reduced. This point also contributes to improvement of the reliability of the chip laminate.

Further, in some embodiments of this invention as described above, the electronic and/or the semiconductor device according to this invention encompass a laminated semiconductor element where said lamination consists of a photosensitive composition according to the present invention.

In some embodiments of this invention, the semiconductor device encompassing a redistribution layer (RDL) structure further incorporates a photosensitive composition according to this invention.

Further, in some embodiments of this invention as described above, the semiconductor device encompassing a chip stack structure further includes a photosensitive composition according to this invention.

In yet some other embodiments of this invention as described above, the semiconductor device encompassing a complementary metal oxide semiconductor (CMOS) image sensor dam structure further incorporates a photosensitive composition according to this invention.

In addition, in some embodiments of this invention as described above, a film is formed by the photosensitive composition according to this invention. As further described above, such films especially after curing generally exhibit excellent chemical, mechanical, elastic properties having a wide variety of utility in electronic, optoelectronic, microelectromechanical applications featuring excellent dielectric properties.

Accordingly, in some embodiments of this invention, there is provided a microelectronic or optoelectronic device encompassing one or more of a redistribution layer (RDL) structure, a chip-stack structure, a CMOS image sensor dam structure, where said structures further incorporates a photosensitive composition according to this invention.

Further, in some embodiments of this invention, there is provided a method of forming a film for the fabrication of a microelectronic or optoelectronic device comprising:
coating a suitable substrate with a composition according to the invention to form a film;
patterning the film with a mask by exposing to a suitable radiation;
developing the film after exposure to form a photo-pattern; and
curing the film by heating to a suitable temperature.

The coating of the substrate with photosensitive composition of this invention can be performed by any of the coating procedures as described herein and/or known to one skilled in the art, such as by spin coating.

In another aspect of this invention there is also provided a cured product comprising the composition of this invention.

This invention is further illustrated by the following examples which are provided for illustration purposes and in no way limit the scope of the present invention.

### EXAMPLES (GENERAL)

The following abbreviations have been used hereinbefore and hereafter in describing some of the compounds, instruments and/or methods employed to illustrate certain of the embodiments of this invention:
ODPA—5,5′-oxybis(isobenzofuran-1,3-dione);
6FDA—5,5′-(perfluoropropane-2,2-diyl)bis(isobenzofuran-1,3-dione);
PMDA—1H,3H-benzo[1,2-c:4,5-c′]difuran-1,3,5,7-tetraone;
6BF—4,4′-([1,1′-biphenyl]-4,4′-diylbis(oxy))bis(3-(trifluoromethyl)aniline);
PFMB—2,2′-bis(trifluoromethyl)-[1,1′-biphenyl]-4,4′-diamine;
APB—4,4′-(1,3-phenylenebis(oxy)) dianiline;
JD230—a diamine of formula (ID) as described herein where n=3;
DO3—4,4′-methylenebis (2,6-dimethylaniline);
DMMIEtNH₂—1-(2-aminoethyl)-3,4-dimethyl-1H-pyrrole-2,5-dione;
2-NH₂PhSH—2-aminobenzenethiol;
PCG-Precursor amine—(4-(3-(trifluoromethyl)-3H-diazirin-3-yl)phenyl)methanamine;
Hunig's Base—N-ethyl-N-isopropylpropan-2-amine;
PCG-1—3,3′-((oxybis(methylene))bis(4,1-phenylene))bis(3-(trifluoromethyl)-3H-diazirine);
PCG-3—5,5′-oxybis(2-(4-(3-(trifluoromethyl)-3H-diazirin-3-yl)benzyl)isoindoline-1,3-dione);
TAEICY—(2,4,6-trioxo-1,3,5-triazinane-1,3,5-triyl)tris(ethane-2,1-diyl) triacrylate;
FC-4432—a non-ionic polymeric fluorochemical surfactant from 3M Advanced Materials;
KBM-403E—trimethoxy(3-(oxiran-2-ylmethoxy)propyl)silane;
TMAH—tetramethylammonium hydroxide;
NMP—N-methyl-2-pyrrolidone;
GBL—γ-butyrolactone;
DMAc—N,N-dimethylacetamide;
DMSO—dimethyl sulfoxide;
THF—tetrahydrofuran;
GPC—gel permeation chromatography;
M_{w}—weight average molecular weight;
Mₙ—number average molecular weight;
PDI—polydispersity index;
¹H-NMR—proton nuclear magnetic resonance spectroscopy;
FT-IR—Fourier transform infrared spectroscopy;
ppm—parts per million;
pphr—parts per hundred parts of resin.

### Example 1

### PMDA/APB/JD230 (50/30/20)

A mixture of APB (17.5 g, 60 mmol) and JD230 (9.5 g, 40 mmol) were dissolved in NMP (196 g) and stirred at ambient temperature under a nitrogen atmosphere. To this solution was then added PMDA (21.8 g, 100 mmol) in small batches while stirring. The reaction mixture was continued to stir at ambient temperature for 20 hours and diluted with cyclopentanone (100 g). The polymer solution was added to 1.5 L water/acetone (80/20) mixture to isolate the polyamic acid. The gummy product was washed with 1 L water/methanol (80/20) mixture and dried in a vacuum oven at 80-90° C. for 24 hours to obtain a solid product (43 g, 85%isolated yield).

### Example 2

### 6FDA/PMDA/6BF/PFMB/DMMIEtNH₂ (24.7/24.7/29/19.1/2.5)

A mixture of 6BF (17.8 g, 35 mmol), PFMB (7.5 g, 23 mmol) and DMMIEtNH₂ (0.5 g, 3 mmol)were dissolved in NMP (182 g) and stirred at ambient temperature under a nitrogen atmosphere. A mixture of 6FDA (13.3 g, 30 mmol) and PMDA (6.5 g, 30 mmol) was added in small batches to the above solution while stirring. The reaction mixture was stirred at ambient temperature for an additional period of 20 hours during which time the solution turned viscous. An additional amount of NMP (62 g) was added to this viscous solution. A small portion of this solution was then diluted with DMAc for GPC analysis. GPC-DMAc—M_{w}=121,150, Mₙ=69,500, PDI=1.74. A small sample of the polymer solution was also added to excess water/acetone (80/20) mixture to isolate the polymer for ¹H NMR analysis. The gummy product was washed with excess water/acetone (80/20) mixture and dried in a vacuum oven at 50-60° C. for 24 hours to obtain a solid product. ¹H-NMR (500 MHz) spectra measured in deuterated DMSO showed a broad peak centered at about 13.5 ppm for COOH and 10.97 ppm, 10.87 ppm, and 10.85 ppm for —NH— groups of the polyamic acid in approximately 1:1 ratio. Multiple peaks were observed at 6.7-8.7 ppm for the aromatic protons from 6FDA, PMDA PFMB and 6BF.

The polyamic acid solution (195 g containing about 31 g polymer) thus obtained was then mixed with anhydrous pyridine (31 g), acetic anhydride (32 g) and cyclopentanone (60 g) and the solution was heated to 95° C. for 6 hours under nitrogen atmosphere while stirring. The reaction mixture was allowed to cool to ambient temperature and THF (50 g) was added. This solution was added to excess water/methanol (80/20) mixture (2.5 L) to isolate the polymer. The gummy product was washed with excess (2.5 L) water/methanol (80/20) mixture and dried in a vacuum oven at 70° C. for 24 hours to obtain a solid product (32 g, quantitative yield), which was characterized by GPC and ¹H NMR. GPC-DMAc—M_{w}=90,350, Mₙ=48,000, PDI=1.9. ¹H-NMR (500 MHz) spectra measured in deuterated DMSO showed only traces of a broad peak centered at about 13.5 ppm for COOH and 10-11 ppm for —NH— groups indicating that the poly(amic acid) was quantitatively imidized. Multiple peaks were observed at 6.7-8.7 ppm for the aromatic protons from 6FDA, PMDA PFMB and 6BF. FT-IR spectra showed peaks at 1375 cm-1 and 723 cm-1 characteristic of polyimides.

### Example 3

### 6FDA/PMDA/6BF/PFMB/2-NH₂PhSH (24.7/24.7/29.0/19.1/2.5)

A mixture of 6BF (14.8 g, 29 mmol), PFMB (6.2 g, 19 mmol) and 2-NH₂PhSH (0.3 g, 2.5 mmol) were dissolved in NMP (152 g) and stirred at ambient temperature under a nitrogen atmosphere. To this solution was added a mixture of 6FDA (11 g, 25 mmol) and PMDA (5.5 g, 25 mmol) in small batches while stirring. The reaction mixture was stirred at ambient temperature for 20 hours during which time the solution turned viscous. An additional amount of NMP (65 g) was added to the viscous solution. A small portion of this solution was then diluted with DMAc for GPC analysis. GPC-DMAC—M_{w}=127,800, Mₙ=70,450, PDI=1.8. A small sample of the polymer solution was added to excess water/acetone (80/20) mixture to isolate the polymer. The gummy product was washed with excess water/acetone (80/20) mixture and dried in a vacuum oven at 50-60° C. for 24 hours to obtain a solid product. ¹H-NMR (500 MHz) spectra measured in deuterated DMSO showed a broad peak centered at about 13.5 ppm for COOH and 10.99 ppm, 10.88 ppm, and 10.86 ppm for —NH— groups of the poly(amic acid) in approximately 1:1 ratio. Multiple peaks in 6.7-8.7 ppm for the aromatic protons from 6FDA, PMDA PFMB and 6BF were also observed.

The polyamic acid solution (129 g containing about 19 g polymer) thus obtained above was mixed with anhydrous pyridine (19 g), acetic anhydride (19 g) and cyclopentanone (85 g) and the solution was heated to 90° C. for 6 hours under nitrogen atmosphere while stirring. The reaction mixture was allowed to cool to ambient temperature and THF (50 g) was added. This solution was added to excess water/methanol (80/20) mixture (2 L) to isolate the polymer. The gummy product was washed with excess (2 L) water/methanol (80/20) mixture and dried in a vacuum oven at 70° C. for 24 hours to obtain a solid product (19 g, 98% isolated yield, GPC-DMAC M_{w}=151,700, Mₙ=69,550, PDI=2.2). ¹H-NMR (500 MHz) spectra measured in deuterated DMSO did not show a broad peak centered at about 13.5 ppm for COOH or 10-11 ppm for —NH— groups indicating that the poly(amic acid) was quantitatively imidized. Multiple peaks in 6.7-8.7 ppm for the aromatic protons from 6FDA, PMDA PFMB and 6BF were also observed.

### Example 4

### 6FDA/PMDA/6BF/DO3/DMMIEtNH₂ (24.6/24.6/28.6/18.8/3.4)

A mixture of 6BF (14.8 g, 29 mmol), DO3 (5 g, 19 mmol) and DMMIEtNH₂ (0.4 g, 2.5 mmol) were dissolved in NMP (147 g) and stirred at ambient temperature under a nitrogen atmosphere. A mixture of 6FDA (11.1 g, 25 mmol) and PMDA (5.5 g, 25 mmol) was added in small batches to the above solution while stirring. The reaction mixture was stirred at ambient temperature for 20 hours during which time the solution turned viscous. An additional amount of NMP (78 g) was added to this viscous solution. A small portion of this solution was then diluted with DMAc for GPC analysis. GPC-DMAc—M_{w}=111,040, Mₙ=64,700, PDI=1.72. A small sample of the polymer solution was added to excess water/acetone (80/20) mixture to isolate the polymer. The gummy product was washed with excess water/acetone (80/20) mixture and dried in a vacuum oven at 50-60° C. for 24 hours to obtain a solid product. ¹H-NMR (500 MHz) spectra measured in deuterated DMSO showed a broad peak centered at about 13.5 ppm for COOH and 10.97 ppm, 10.87 ppm, and 10.85 ppm for —NH— groups of the poly(amic acid) in approximately 1:1 ratio. Multiple peaks in 6.7-8.7 ppm for the aromatic protons from 6FDA, PMDA PFMB and 6BF were also observed.

The polyamic acid solution (200 g containing about 28 g polymer) thus obtained above was mixed with anhydrous pyridine (28 g), acetic anhydride (29 g) and cyclopentanone (81 g) and the solution heated to 90° C. for 5 hours under nitrogen atmosphere while stirring. The reaction mixture was allowed to cool to ambient temperature and THF (100 g) was added. This solution was added to excess water/acetone (80/20) mixture (2.5 L) to isolate the polymer. The gummy product was washed with excess (2.5 L) water/acetone (80/20) mixture and dried in a vacuum oven at 80-90° C. for 24 hours to obtain a solid product (26.8 g, 96% yield, GPC-DMAC M_{w}=94,200, Mₙ=47,000, PDI=2). ¹H-NMR (500 MHz) spectra measured in deuterated DMSO showed only traces of a broad peak centered at about 13.5 ppm for COOH and 10-11 ppm for —NH— groups indicating that the poly(amic acid) was quantitatively imidized. Multiple peaks in 6.7-8.7 ppm for the aromatic protons from 6FDA, PMDA PFMB and 6BF were observed. FT-IR spectra showed peaks at 1373 cm⁻¹ and 723 cm⁻¹ characteristic of polyimides.

### Example 5

### 6FDA/PMDA/6BF/PFMB (25/25/30/20)

A mixture of 6BF (15.1 g, 30 mmol) and PFMB (6.4 g, 20 mmol) were dissolved in NMP (152 g) and stirred at ambient temperature under a nitrogen atmosphere. To this solution was added a mixture of 6FDA (11.1 g, 25 mmol) and PMDA (5.5 g, 25 mmol) in small batches while stirring. The reaction mixture was continued to stir at ambient temperature for 20 hours during which time the solution turned viscous. A small amount of this solution was diluted with DMAc for GPC analysis. GPC-DMAc—M_{w}=258,950, Mₙ=124,450, PDI=2.1.

The polyamic acid solution thus formed (236 g containing 23.6 g polymer) was mixed with anhydrous pyridine (24 g), acetic anhydride (24 g) and cyclopentanone (61 g) and the solution heated to 95° C. for 4 hours under nitrogen atmosphere while stirring. The reaction mixture was allowed to cool to ambient temperature and added to excess water/methanol (80/20) mixture (2 L) to isolate the polymer. The gummy product was washed with excess (2 L) water/methanol (80/20) mixture and dried in a vacuum oven at 70° C. for 24 hours to obtain a solid product (23 g, 97% isolated yield, GPC-DMAC M_{w}=275,750, Mₙ=114,750, PDI=2.4). ¹H-NMR (500 MHz) spectra measured in deuterated DMSO showed only traces of a broad peak centered at about 13 ppm for COOH and 11.13 ppm, 10.99 ppm and 10.86 ppm for —NH— groups of the poly(amic acid) indicating that the polyamic acid was quantitatively imidized. Multiple peaks in 6.7-8.6 ppm for the aromatic protons for 6FDA, PMDA, 6BF and PFMB were also observed. FT-IR spectra showed peaks at 1375 cm⁻¹ and 723 cm⁻¹ characteristic of polyimides.

### Example 6

### 6FDA/PMDA/6BF/PFMB/DMMIEtNH₂ (24.6/24.6/28.6/18.8/3.4)

A mixture of 6BF (14.7 g, 29 mmol), PFMB (6.1 g, 19 mmol) and DMMIEtNH₂ (0.6 g, 3.5 mmol) were dissolved in NMP (152 g) and stirred at ambient temperature under a nitrogen atmosphere. To this solution was added a mixture of 6FDA (11.1 g, 25 mmol) and PMDA (5.5 g, 25 mmol) in small batches while stirring. The reaction mixture was stirred at ambient temperature for 20 hours during which time the solution had turned viscous. An additional amount of NMP (69 g) was added to this viscous solution (GPC-DMAC M_{w}=111,042, Mₙ=64,700, PDI=1.7). ¹H-NMR (500 MHz) spectra of the reaction mixture measured in deuterated DMSO showed peaks at 10.87 ppm, 10.87 ppm, and 10.85 ppm for —NH— groups of the poly(amic acid). Multiple peaks in 6.7-8.7 ppm for the aromatic protons from 6FDA, PMDA PFMB and 6BF were also observed.

The polyamic acid solution thus formed above (200 g containing about 30 g polymer) was mixed with anhydrous pyridine (33 g), acetic anhydride (30 g) and cyclopentanone (33 g) and the solution was heated to 95° C. for 6 hours under nitrogen atmosphere while stirring. The reaction mixture was allowed to cool to ambient temperature and THF (25 g) was added. This solution was added to excess water/methanol (80/20) mixture (2.5 L) to isolate the polymer. The gummy product was washed with excess (2 L) water/methanol (80/20) mixture and dried in a vacuum oven at 80-90° C. for 24 hours to obtain a solid product (28 g, 93% yield, GPC-DMAC M_{w}=94,200, Mₙ=47,000, PDI=2). ¹H-NMR (500 MHz) spectra measured in deuterated DMSO showed only traces of peaks at 10.5-11.5 ppm for —NH— groups indicating that the poly(amic acid) was quantitatively imidized. Multiple peaks in 6.7-8.7 ppm for the aromatic protons from 6FDA, PMDA PFMB and 6BF were observed. FT-IR spectra showed peaks at 1373 cm⁻¹ and 723 cm⁻¹ characteristic of polyimides.

### Example 7

### Synthesis of PCG-2, a Precursor to PCG-3

A mixture of the hydrochloric acid salt of the PCG-Precursor amine (1 g, 4 mmol), ODPA (0.62 g, 2 mmol), Hunig's Base (0.52 g, 4 mmol) dissolved in anhydrous THF (10 g) was stirred at ambient temperature for 24 hours. To this mixture was added DMSO (5 g) to dissolve any remaining solids and the stirring was continued for additional 20 hours. To this reaction mixture water (100 g) was added to separate a colorless solid. This solid was washed with water (100 g) and filtered. The product was dried in a vacuum oven at ambient temperature for 24 hours to obtain PCG-2 (1.3 g, 81% isolated yield). Liquid Chromatography-Mass Spectroscopy (LC-MS) analysis performed in methanol confirmed the formation of the desired product. LC-MS, m/z=739 (negative mode ionization), m/z=741 (positive mode ionization), exact mass of PCG-2=740. ¹³C-NMR carried out in deuterated DMSO showed a quartet at 28 ppm, 28.3 ppm, 28.7 ppm and 28.9 ppm characteristic of the carbon attached to diazirine and trifluoromethyl groups. A broad peak at 12-13 ppm was observed in the ¹H-NMR spectrum taken in deuterated DMSO indicating the presence of —COOH groups as a result of the ring opening of ODPA by the PCG-Precursor amine.

### Example 8

### Synthesis of PCG-3

A mixture of PCG-2 (0.6 g, 0.81 mmol), pyridine (0.13 g, 1.6 mmol) and acetic anhydride (0.16 g, 1.6 mmol) dissolved in DMSO (2 g) was stirred at ambient temperature for 24 hours. To this reaction mixture was added water (5 g) to separate the title compound as a solid. The solid product was washed with water (2×25 g) and dried in a vacuum oven at ambient temperature for 24 hours (0.4 g, 65% isolated yield). LC-MS of this product performed in methanol, m/z=361, exact mass of PCG-3=704. The detected peak corresponds to a fragmentation ion product (PCG-3 Fragment).

### Examples 9-11

### Photo Imaging Studies Using Polyamic Acid With Aqueous Base Development

The polyamic acid prepared in accordance with the procedures of Example 1 (PMDA/APB/JD230, 50/30/20) was dissolved in GBL/cyclopentanone (3:1 wt. ratio) at 22 wt. % solution and was used to prepare the compositions of Examples 9-11. To each of these compositions was added PCG-1, various levels as summarized in Table 1, adhesion promoter (KBM-403E, 5 pphr) and surface leveling agent (FC-4432, 0.3 pphr). The composition of Example 11 also contained an epoxy cross linker (TMPTGE).

The compositions so formed in each of Examples 9-11 and the composition from Comparative Example 1 were filtered using 0.2 μm pore polytetrafluoroethylene (PTFE) disc filter and stored refrigerated before use. Table 1 summarizes the amounts of PCG-1, TMPTGE and TAEICY, if used, in each of the compositions of Examples 9 to 11 and the Comparative Example 1.

TABLE 1

| Example No. | PCG-1 | TMPTGE | TAEICY |
|---|---|---|---|
| Example 9 | 30 pphr | — | — |
| Example 10 | 20 pphr | — | — |
| Example 11 | 20 pphr | 20 pphr | — |
| Comp. Ex. 1 | 20 pphr | — | 20 pphr |

Each of the compositions from Examples 9 to 11 and the Comparative Example 1 were then spin coated on 4″ SiO₂ wafers by spin coating for 30 seconds followed by post apply bake (PAB) at 100° C. for 3 minutes to generate films of about 3-6 μm thickness. The films were then exposed using a broad band Hg-vapor light source (at 365 nm using a band pass filter) at an exposure dose ranging from 0-1500 mJ/cm² through a mask to generate lines, trenches, pillars and holes. The exposed film was post exposure baked (PEB) at 100° C. for 2 minutes and developed with CD-26 (2.38 wt. % TMAH) immersion to reveal the patterns corresponding to the mask. The film thicknesses of the exposed areas were measured at various exposure doses to determine the film thickness loss (bright field loss, BFL). Table 2 summarizes the imaging results. The resolution of features such as lines (L), square pillars (P) and vias (V) are given in micrometers. It is evident from this data that use of PCG-1 enables the compositions prepared in accordance of this invention to photo image with aqueous base development. It is even more important and surprising to note that the thickness loss (BFL) is very low for compositions containing no additional additives such as the epoxy or the acrylate crosslinker. It is further surprising to note that the thickness loss is most significant for the composition of Comparative Example 1 suggesting that acrylate crosslinkers are not suitable for the compositions of this invention.

TABLE 2

| | FT | Dose | BFL | Resolution |
|---|---|---|---|---|
| Example No. | (μm) | (mJ/cm²) | (%) | L/P/V |
| Example 9 | 4.46 | 608 | 12 | 20/20/70 |
| Example 10 | 3.83 | 1187 | 0 | 20/40/30 |
| Example 11 | 5.24 | 1187 | 15 | 15/30/30 |
| Comp. Ex. 1 | 4.56 | 1187 | 41 | 15/15/40 |

FT—film thickness;
BFL—bright field loss;
L—lines;
P—pillars;
V—vias

### Examples 12-14

### Photo Imaging Studies Using Polyamic Acid and Polyimides With Cyclopentanone Development

The polyamic acid prepared in accordance with the procedures as set forth in Example 2 (PMDA/6FDA/6BF/PFMB/DMMI, 24.7/24.7/29.0/19.1/2.5) was dissolved in NMP at 15 wt. % solution and was used to prepare the composition of Example 12. The polyamic acid prepared in accordance with the procedures as set forth in Example 3 (PMDA/6FDA/6BF/PFMB/2-NH₂PhSH, 24.7/24.7/29.0/19.1/2.5) was dissolved in NMP at 15 wt. % solution and was used to prepare the composition of Example 13. The polyamic acid prepared in accordance with the procedures as set forth in Example 4 (PMDA/6FDA/6BF/DO3/DMMI, 24.7/24.7/29.0/19.1/2.5) was dissolved in NMP at 15 wt. % solution and was used to prepare the composition of Example 14. To each of these solutions were added photo carbene generator (PCG-1, 25 pphr), adhesion promoter (KBM-403E, 5 pphr), and surface leveling agent (FC-4432, 0.3 pphr).

The compositions so formed in each of Examples 12 to 14 and the compositions of Comparative Examples 2 to 4, which contained the corresponding polyimides, were filtered using 0.2 μm pore polytetrafluoroethylene (PTFE) disc filter and stored refrigerated before use.

Each of these compositions were then spin coated on 4″ SiO₂ wafers by spin coating at 375-1500 rpm for 30 seconds followed by post apply bake (PAB) at 100° C. for 3 minutes to generate films of about 2-4 μm thickness. The films were then exposed using a broad band Hg-vapor light source (at 365 nm using a band pass filter) at an exposure dose ranging from 0-1500 mJ/cm² through a mask to generate lines, trenches, pillars and holes. The exposed film was post exposure baked (PEB) at 100° C. for 2 minutes and developed with cyclopentanone immersion to reveal the patterns corresponding to the mask. The film thicknesses of the exposed areas were measured at various exposure doses to determine the film thickness loss (bright field loss, BFL). Table 3 summarizes the imaging results. The resolution of features such as lines (L), square pillars (P) and vias (V) are given in micrometers. It is evident from this data that use of PCG-1 enables various poly-amic acid compositions to photo image with solvent development. However corresponding polyimides are not amenable to imaging since those polymers do not contain —COOH groups so that the photo carbene generated can insert into the —COOH group of the amic acid and form a cross linked network. Examples 14A-C show the BFL and resolution at three exposure doses of the same wafer processed.

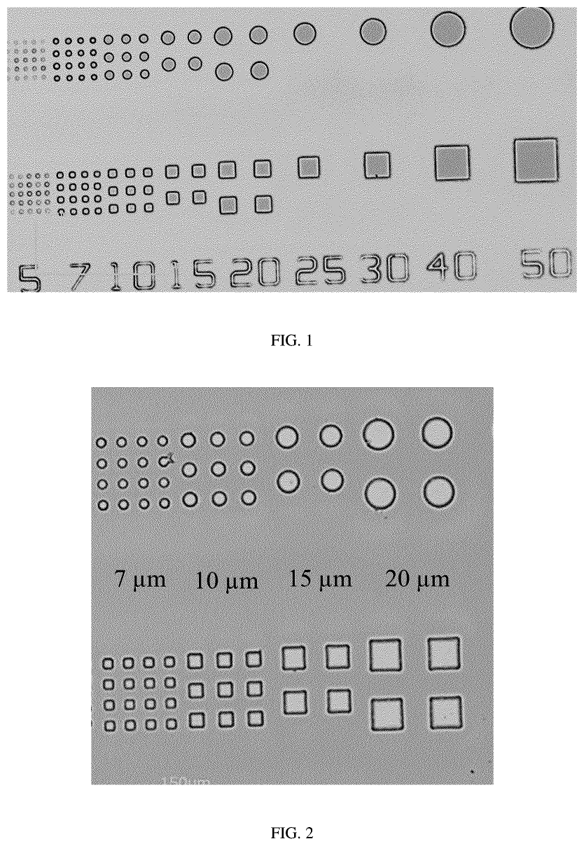

FIG. 1 shows the formation of square and circular vias from Example 12 at 7-50 μm resolution. FIG. 2 shows the formation of square and circular pillars from Example 12 at 7-20 μm resolution.

TABLE 3

| | Spin | | Dose | Develop | | |
|---|---|---|---|---|---|---|
| | Speed | FT | (mJ/ | Time | BFL | Resolution |
| Example No. | (rpm) | (μm) | cm²) | (sec) | (%) | L/P/V |
| Example 12 | 500 | 2.03 | 1187 | 30 | 0.5 | 7/7/7 |
| Example 13 | 350 | 2.39 | 1187 | 20 | 0 | 7/5/7 |
| Example 14A | 700 | 2.77 | 966 | 60 | 0 | 7/5/7 |
| Example 14B | 700 | 2.77 | 765 | 60 | 5 | 7/5/7 |
| Example 14C | 700 | 2.77 | 607 | 60 | 14 | 7/7/7 |
| Comp. Ex. 2 | 1000 | 2.62 | 1500 | 30 | 75 | Poor image |
| Comp. Ex. 3 | 1500 | 2.99 | 1500 | 20 | 11 | Poor image |
| Comp. Ex. 4 | 700 | 3.99 | 1500 | 30 | 10 | Poor image |

FT—film thickness;
BFL—bright field loss;
L—lines;
P—pillars;
V—vias

### Example 15

The polyamic acid prepared in accordance with the procedures as set forth in Example 2 (PMDA/6FDA/6BF/PFMB/DMMI, 24.7/24.7/29.0/19.1/2.5) was dissolved in NMP at 12.5 wt. % solution and was used to prepare the composition of Example 15. To this solution was added photo carbene generator (PCG-3, 20 pphr), an adhesion promoter (KBM-403E, 5 pphr), and surface leveling agent (FC-4432, 0.3 pphr). This properties of this composition was then compared with the composition of Comparative Example 5, which contained photo carbene generator (PCG-2, 20 pphr) as the crosslinking agent.

The compositions so formed in Example 15 and Comparative Example 5 were filtered using 0.2 μm pore polytetrafluoroethylene (PTFE) disc filter and stored refrigerated before use.

Each of these compositions were then spin coated on 4″ SiO₂ wafers by spin coating at 800 rpm for 30 seconds followed by post apply bake (PAB) at 100° C. for 3 minutes to generate films of about 4 um thickness. The films were then exposed using a broad band Hg-vapor light source (at 365 nm using a band pass filter) at an exposure dose of 2000 mJ/cm² through a mask to generate lines, trenches, pillars and holes. The exposed films were post exposure baked (PEB) at 100° C. for 2 minutes and developed with cyclopentanone immersion to reveal the patterns corresponding to the mask. The film thicknesses of the exposed areas were measured to determine the film thickness loss (bright field loss, BFL). Table 4 summarizes the imaging results. It is evident from this data that use of PCG-3 with a ring closed structure enables various poly-amic acid compositions to photo image with solvent development. However corresponding ring opened version (PCG-2) is not amenable to imaging since this bisdiazirine contains —COOH groups which may undergo unimolecular carbene insertion thereby preventing insertions to the —COOH groups of the polyamic acid polymer.

TABLE 4

| | FT | Development | BFL | |
|---|---|---|---|---|
| Example No. | (μm) | (sec) | (%) | Imaging |
| Example 15 | 4.25 | 105 | 25 | Yes |
| Comp. Ex. 5 | 4.03 | 60 | 100 | No |

FT—film thickness;
BFL—bright field loss

### Examples 16-19

The polyamic acid prepared in accordance with the procedures as set forth in Example 2 (PMDA/6FDA/6BF/PFMB/DMMI, 24.7/24.7/29.0/19.1/2.5) was dissolved in NMP at 15 wt. % solution and was used to prepare the composition of Example 16. The polyamic acid prepared in accordance with the procedures as set forth in Example 5 (6FDA/PMDA/6BF/PFMB, 25/25/30/20) was dissolve in NMP at 15 wt. % solution and was used to prepare the composition of Example 17. The polyamic acid prepared in accordance with the procedures as set forth in Example 6 (6FDA/PMDA/6BF/PFMB/DMMIEtNH₂, 24.6/24.6/28.6/18.8/3.4) was used to prepare the compositions of Examples 18 and 19. To these solutions were added photo carbene generator (PCG-1, 25 pphr) for Examples 16-18 and photo carbene generator (PCG-3, 25 pphr) for Example 19. Each of the compositions further contained adhesion promoter (KBM-403E, 5 pphr), surface leveling agent (FC-4432, 0.3 pphr) and GBL (100 pphr) as an extra solvent.

The compositions so formed in Examples 16-19 were filtered using 0.2 μm pore polytetrafluoroethylene (PTFE) disc filter and stored refrigerated before use.

These compositions were then spin coated on 4″ SiO₂ wafers by spin coating at 1500 rpm for 30 seconds for Example 16 and 2300 rpm for 30 seconds for Examples 17-19 followed by post apply bake (PAB) at 95° C. for 3 minutes to generate films of about 1-3 μm thickness. The films were then exposed using a broad band Hg-vapor light source (at 365 nm using a band pass filter) at an exposure dose of 0-1000 mJ/cm² through a mask to generate lines, trenches, pillars and holes. The exposed films were post exposure baked (PEB) at 95° C. for 2 minutes and developed with cyclopentanone immersion for 60 seconds to reveal the patterns corresponding to the mask. The film thicknesses of the exposed areas were measured at various exposure doses to determine the film thickness loss (bright field loss, BFL). Table 5 summarizes the imaging results. It is evident from this data that use of PCG-1 or PCG-3 enables various poly-amic acid compositions of this invention to photo image with solvent development.

TABLE 5

| | FT | Dose | BFL | Resolution |
|---|---|---|---|---|
| Example No. | (μm) | (mJ/cm²) | (%) | (LP/V) |
| Example 16 | 1.33 | 765 | 14 | 10/10/10 |
| Example 17 | 2.92 | 491 | 16 | 10/15/20 |
| Example 18 | 2.15 | 765 | 8 | 10/15/20 |
| Example 19 | 2.71 | 966 | 39 | 7/15/20 |

FT—film thickness;
BFL—bright field loss;
L—lines;
P—pillars;
V—vias

### Examples 20-21

### Thermomechanical Properties

Each of the compositions from Example 10, Example 11 and Comparative Example 1 were spin coated on 5″ bare silicon wafers and post apply baked (PAB) at 100° C. for 3 minutes to generate films at about 8-10 μm thick. The films were then exposed using a broad band Hg-vapor light source (at 365 nm using a band pass filter) at an exposure dose of 1000 mJ/cm² and post exposure baked (PEB) at 100° C. for 2 minutes. The films were cured at 170° C. for 4 hours in an oven under nitrogen atmosphere. These cured films were diced into 6 mm wide rectangular strips for thermo-mechanical property measurements by Instron and Thermal Mechanical Analysis (TMA). The results are summarized in Table 6. It is evident from the data presented in Table 6, the film made from the composition of Example 10 which contained PCG-1 (Example 20) is capable of having a low coefficient of expansion (CTE) and high tensile strength, which also exhibited high glass transition temperature (T_{g}). Similarly, the film made from the composition of Example 11, which contained epoxy crosslinker along with PCG-1 (Example 21) exhibited high elongation to break and high Young's modulus.

TABLE 6

| | | Tensile | | Young's | CTE | |
|---|---|---|---|---|---|---|
| Composition | | Strength | ETB | Modulus | (ppm/ | T_{g} |
| Example No. | Example No. | (MPa) | (%) | (GPa) | K) | (° C.) |
| Example 10 | Example 20 | 119 | 6 | 2.99 | 36 | 194 |
| Example 11 | Example 21 | 110 | 20 | 3.33 | 91 | 170 |
| Comp. Ex. 1 | Comp. Ex. 6 | 116 | 5 | 2.80 | 60 | 188 |

ETB—elongation to break;
CTE—coefficient of thermal expansion;
T_{g}—glass transition temperature

On the other hand the film made from the Comparative Example 1, which contained acrylate crosslinker, TAEICY, along with PCG-1 (Comparative Example 6) exhibited lower Young's modulus as well as low elongation to break. These results clearly demonstrate that the compositions of this invention exhibits superior photoimaging as well as thermo-mechanical properties even in the absence of conventional epoxy and/or acrylate crosslinkers.

### Comparative Example 1

The polyamic acid prepared in accordance with the procedures of Example 1 (PMDA/APB/JD230, 50/30/20) was dissolved in GBL/cyclopentanone (3:1 wt. ratio) at 22 wt. % solution. To this solution was added PCG-1 (20 pphr), adhesion promoter (KBM-403E, 5 pphr), surface leveling agent (FC-4432, 0.3 pphr) and TAEICY (20 pphr). The composition thus formed was used in photoimaging studies and compared with the compositions prepared in accordance of this invention as set forth in Examples 9 to 11.

### Comparative Examples 2 to 4

The polyimide prepared in accordance of the procedures as set forth in Example 2 was dissolved in GBL/cyclopentanone mixture (3:1 wt. ratio) at 15 wt. % solution and was used to form the composition of Comparative Example 2. The polyimide prepared in accordance of the procedures as set forth in Example 3 was dissolved in GBL/cyclopentanone mixture (3:1 wt. ratio) at 15 wt. % solution and was used to form the composition of Comparative Example 3. The polyimide in accordance of the procedures as set forth in Example 4 was dissolved in GBL/cyclopentanone mixture (3:1 wt. ratio) at 15 wt. % solution and was used to form the composition of Comparative Example 4. To each of these solutions were added photo carbene generator (PCG-1, 25 pphr), adhesion promoter (KBM-403E, 5 pphr), and surface leveling agent (FC-4432, 0.3 pphr). The compositions thus formed were used as Comparative Examples 2 to 4 along with the compositions of Examples 12 to 14 for photoimaging studies. The results are summarized in Table 3.

### Comparative Example 5

The polyamic acid prepared in accordance with the procedures as set forth in Example 2 (PMDA/6FDA/6BF/PFMB/DMMI, 24.7/24.7/29.0/19.1/2.5) was dissolved in NMP at 12.5 wt. % solution and was used to prepare the composition of Example 15. To this solution was added photo carbene generator (PCG-2, 20 pphr), an adhesion promoter (KBM-403E, 5 pphr), and surface leveling agent (FC-4432, 0.3 pphr). The properties of this composition is compared with that of the composition of Example 15 and the results are summarized in Table 4.

### Comparative Example 6

The composition of Comparative Example 1 was used to form a film and its thermo-mechanical properties were measured as set forth in Examples 20 to 21. The results are summarized in Table 6.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A composition comprising:
a) a polyamic acid selected from the group consisting of a polyamic acid of formula (LA), an end capped polyamic acid of formula (IB) and an end capped polyamic acid of formula (IC): 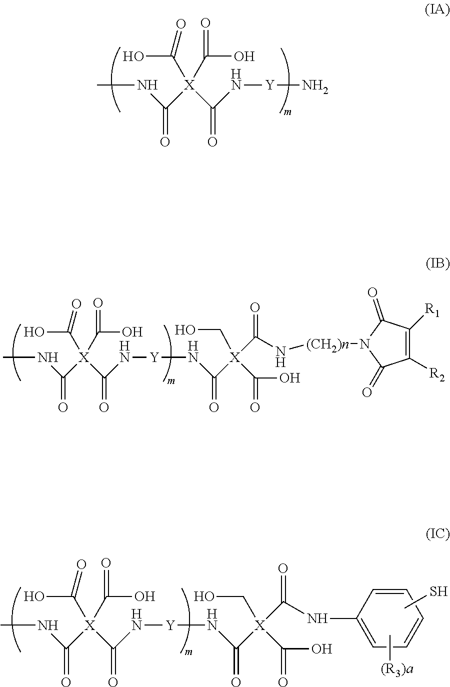
wherein:
m is an integer of at least 50;
n is an integer from 1 to 12, inclusive;
a is an integer from 1 to 4;
X is one or more distinct tetravalent organic group, which is derived from one or more dianhydrides selected from the group consisting of: 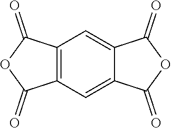 [SMILES: O=c1oc(=O)c2cc3c(=O)oc(=O)c3cc12]
1H,3H-benzo[1,2-c:4,5-c′]difuran-1,3,5,7-tetraone (PMDA); 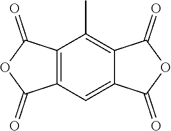 [SMILES: Cc1c2c(=O)oc(=O)c2cc2c(=O)oc(=O)c12]
4-methyl-1H,3H-benzo[1,2-c:4,5-c′]difuran-1,3,5,7-tetraone; 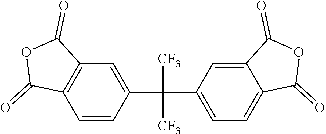 [SMILES: O=C1OC(=O)c2cc(C(c3ccc4c(c3)C(=O)OC4=O)(C(F)(F)F)C(F)(F)F)ccc21]
5,5′-(perfluoropropane-2,2-diyl)bis(isobenzofuran-1,3-dione) (6FDA); 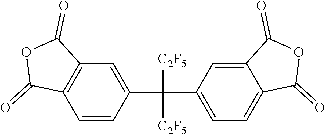
5,5′-(perfluoropentane-3,3-diyl)bis(isobenzofuran-1,3-dione); 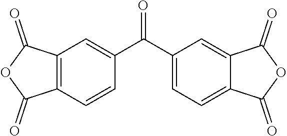 [SMILES: O=C(c1ccc2c(c1)C(=O)OC2=O)c1ccc2c(c1)C(=O)OC2=O]
5,5′-carbonylbis(isobenzofuran-1,3-dione) (BTDA); 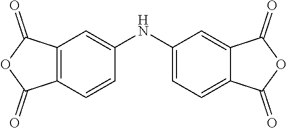 [SMILES: O=C1OC(=O)c2cc(Nc3ccc4c(c3)C(=O)OC4=O)ccc21]
5,5′-azanediylbis(isobenzofuran-1,3-dione); 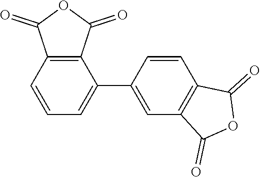 [SMILES: O=C1OC(=O)c2cc(-c3cccc4c3C(=O)OC4=O)ccc21]
[4,5′-biisobenzofuran]-1,1′,3,3′-tetraone (α-BPDA); 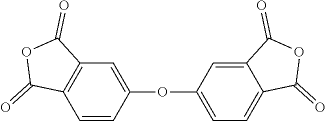 [SMILES: O=C1OC(=O)c2cc(Oc3ccc4c(c3)C(=O)OC4=O)ccc21]
5,5°-oxybis(isobenzofuran-1,3-dione) (ODPA); 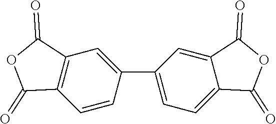 [SMILES: O=C1OC(=O)c2cc(-c3ccc4c(c3)C(=O)OC4=O)ccc21]
[5,5′-biisobenzofuran]-1,1′,3,3′-tetraone (BPDA); and 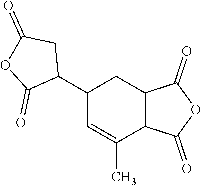 [SMILES: CC1=CC(C2CC(=O)OC2=O)CC2C(=O)OC(=O)C12]
5-(2,5-dioxotetrahydrofuran-3-yl)-7-methyl-3a,4,5,7a-tetrahydroisobenzofuran-1,3-dione (D1901);
Y is one or more distinct divalent organic group derived from a diamine; and
R₁ and R₂ are the same or different and each independently of one another selected from the group consisting of hydrogen, linear or branched (C₁-C₁₆)alkyl, perfluoro(C₁-C₁₂)alkyl, linear or branched (C₁-C₁₆)alkenyl, perfluoro(C₁-C₁₂)alkenyl, (C₆-C₁₀)aryl and (C₆-C₁₀)aryl(C₁-C₃)alkyl; or
R₁ and R₂ taken together with the carbon atoms to which they are attached to form a 5 to 7 membered monocyclic ring, a 6 to 12 membered bicyclic ring or a 9 to 14 membered tricyclic ring, said rings optionally containing one or more heteroatoms selected from O, N and S, and said rings optionally substituted with one or more groups selected from the group consisting of methyl, ethyl, linear or branched (C₃-C₈)alkyl, (C₆-C₁₀)aryl, halogen, hydroxy, linear or branched (C₁-C₈)alkoxy and (C₆-C₁₀)aryloxy; and
each of R₃ is independently selected from the group consisting of hydrogen, halogen, hydroxy, methyl, ethyl, linear or branched (C₃-C₆)alkyl, trifluoromethyl, pentafluoroethyl, linear or branched perfluoro(C₃-C₆)alkyl, methoxy, ethoxy, linear or branched (C₃-C₆)alkyloxy, (C₂-C₆)acyl, (C₂-C₆)acyloxy, phenyl and phenoxy; and
b) a diazirine compound of formula (II): 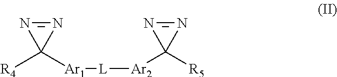
wherein,
L is a bond or a divalent linking or a spacer group selected from the group consisting of: —O—, —O—, —CH₂—, —CHR₆—, —C(R₆)₂—, —C(O)O—R₆—OC(O)—, —C(O)O—R₆—, —R₆—OC(O)—R₆—, —C(O)—R₆—OC(O)—, —C(O)—R₆—, —R₆—C(O)—R₆—, —O—R₆—OC(O)—, —O—R₆—O—, —O—R₆—, —R₆—O—R₆—, —C(O)NH—(CH₂)_{b}—NH(CO)—, where b is an integer from 1 to 15, inclusive, —C(O)NH—(CH₂CH₂O)_{c}(CH₂)ₐ—NR₆(CO)—, where c is an integer from 2 to 6, inclusive and d is an integer from 1 to 6, inclusive, and each occurrence of R₆ may be the same or different which is a divalent group independently selected from the group consisting of methyl, ethyl, linear or branched (C₃-C₁₂)alkyl, (C₃-C₁₂)cycloalkyl, (C₆-C₁₂)aryl, (C₆-C₁₂)aryl(C₁-C₁₂)alkyl, (C₆-C₁₀)heteroaryl, (C₆-C₁₀)heteroaryl(C₁-C₁₂)alkyl, —(CH₂—CH₂—O)ₐ—, where a is an integer from 1 to 10, inclusive;
R₄ and R₅ are the same or different and each independently selected from the group consisting of methyl, ethyl, linear or branched (C₃-C₁₂)alkyl, where portions of hydrogen on alkyl are replaced with fluorine, (C₁-C₁₂)perfluoroalkyl, (C₆-C₁₂)aryl, (C₆-C₁₂)aryl(C₁-C₁₂)alkyl, where portions of hydrogen on alkyl are replaced with fluorine, and (C₆-C₁₂)arylperfluoro(C₁-C₁₂)alkyl; and
Ar₁ and Ar₂ are the same or different and each independently selected from the group consisting of (C₆-C₁₄)arylene, (C₆-C₁₂)heteroarylene, (C₆-C₁₄)aryl(C₁-C₈)alkylene, (C₆-C₁₂)heteroaryl(C₁-C₈)alkylene optionally substituted with a group selected from the group consisting of halogen, —OH, methyl, ethyl, linear or branched (C₃-C₆)alkyl, (C₁-C₄)alkoxy, (C₆-C₁₀)aryl, (C₆-C₁₂)aryloxy, (C₆-C₁₂)aryl(C₁-C₄)alkyl and (C₆-C₁₂)aryl(C₁-C₄)alkyloxy.

2. The composition according to claim 1, wherein X is derived from one or more dianhydrides selected from the group consisting of: 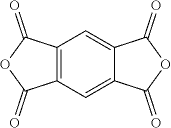 [SMILES: O=c1oc(=O)c2cc3c(=O)oc(=O)c3cc12]
1H,3H-benzo[1,2-c:4,5-c′]difuran-1,3,5,7-tetraone (PMDA): 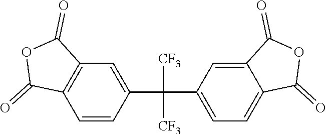 [SMILES: O=C1OC(=O)c2cc(C(c3ccc4c(c3)C(=O)OC4=O)(C(F)(F)F)C(F)(F)F)ccc21]
5,5′-(perfluoropropane-2,2-diyl)bis(isobenzofuran-1,3-dione) (6FDA); and 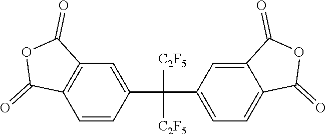
5,5′-(perfluoropentane-3,3-diyl)bis(isobenzofuran-1,3-dione).

3. The composition according to claim 1, wherein Y is derived from one or more diamines selected from the group consisting of: 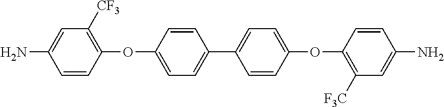 [SMILES: Nc1ccc(Oc2ccc(-c3ccc(Oc4ccc(N)cc4C(F)(F)F)cc3)cc2)c(C(F)(F)F)c1]
4,4′-([1,1′-biphenyl]-4,4′-diylbis(oxy))bis(3-(trifluoromethyl)aniline) (6BF); 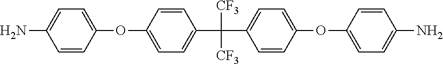 [SMILES: Nc1ccc(Oc2ccc(C(c3ccc(Oc4ccc(N)cc4)cc3)(C(F)(F)F)C(F)(F)F)cc2)cc1]
4,4′-(((perfluoropropane-2,2-diyl)bis(4,1-phenylene))bis(oxy))dianiline (HFBAPP); 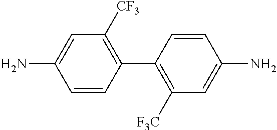 [SMILES: Nc1ccc(-c2ccc(N)cc2C(F)(F)F)c(C(F)(F)F)c1]
2,2′-bis(trifluoromethyl)-[1,1′-biphenyl]-4,4′-diamine (PFMB); 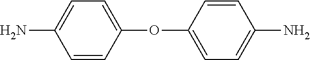 [SMILES: Nc1ccc(Oc2ccc(N)cc2)cc1]
4,4′-oxydianiline (4,4′-ODA); 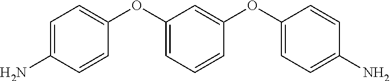 [SMILES: Nc1ccc(Oc2cccc(Oc3ccc(N)cc3)c2)cc1]
4,4′-(1,3-phenylenebis(oxy))dianiline (APB); 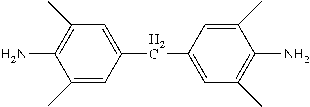 [SMILES: Cc1cc(Cc2cc(C)c(N)c(C)c2)cc(C)c1N]
4,4′-methylenebis(2,6-dimethylaniline) (DO3); 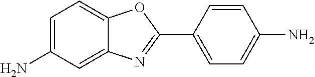 [SMILES: Nc1ccc(-c2nc3cc(N)ccc3o2)cc1]
2-(4-aminophenyl)benzo[d]oxazol-5-amine (BZXPh-5); 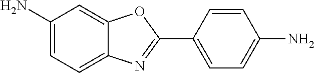 [SMILES: Nc1ccc(-c2nc3ccc(N)cc3o2)cc1]
2-(4-aminophenyl)benzo[d]oxazol-6-amine (BZXPh-6); 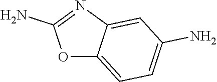 [SMILES: Nc1ccc2oc(N)nc2c1]
benzo[d]oxazole-2,5-diamine (BZX-5); 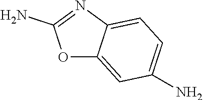 [SMILES: Nc1ccc2nc(N)oc2c1]
benzo[d]oxazole-2,6-diamine (BZX-6); 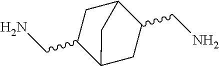 [SMILES: NCC1CC2CC1CC2CN]
bicyclo[2.2.1]heptane-2,5-diyldimethanamine (NBDA);
a diamine of formula (ID) 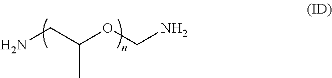
where, n=2 to 6 (JD230); 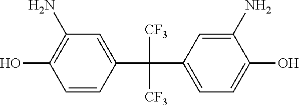 [SMILES: Nc1cc(C(c2ccc(O)c(N)c2)(C(F)(F)F)C(F)(F)F)ccc1O]
4,4′-(perfluoropropane-2,2-diyl)bis(2-aminophenol) (BAFA); 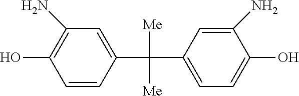 [SMILES: CC(C)(c1ccc(O)c(N)c1)c1ccc(O)c(N)c1]
4,4′-(propane-2,2-diyl)bis(2-aminophenol) (DABPA); and 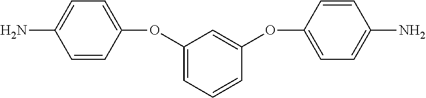 [SMILES: Nc1ccc(Oc2cccc(Oc3ccc(N)cc3)c2)cc1]
4,4′-(1,3-phenylenebis(oxy))dianiline (TPE-R).

4. The composition according to claim 1, wherein the polyamic acid of formula (IB) is end capped with a compound of formula (III): 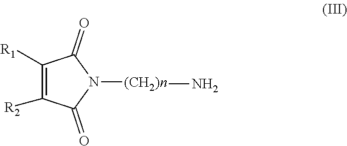
wherein n, R₁ and R₂ are as defined in claim 1.

5. The composition according to claim 4, wherein the compound of formula (III) is selected from the group consisting of: 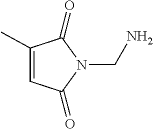
1-(aminomethyl)-3-methyl-1H-pyrrole-2,5-dione; 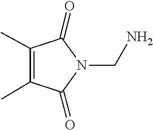
1-(aminomethyl)-3,4-dimethyl-1H-pyrrole-2,5-dione; 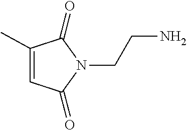
1-(2-aminoethyl)-3-methyl-1H-pyrrole-2,5-dione; 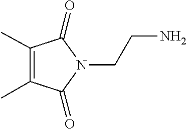
1-(2-aminoethyl)-3,4-dimethyl-1H-pyrrole-2,5-dione (DMMIEtNH₂); 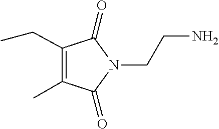
1-(2-aminoethyl)-3-ethyl-4-methyl-1H-pyrrole-2,5-dione; and 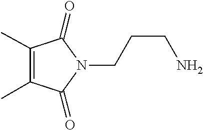
1-(3-aminopropyl)-3,4-dimethyl-1H-pyrrole-2,5-dione.

6. The composition according to claim 1, wherein the polyamic acid of formula (IC) is end capped with a compound of formula (IV): 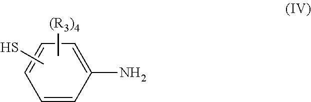
wherein R₃ is as defined in claim 1.

7. The composition according to claim 6, wherein the compound of formula (IV) is selected from the group consisting of: 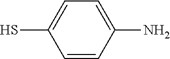
4-aminobenzenethiol; 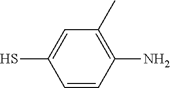
4-amino-3-methylbenzenethiol; 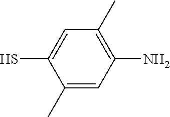
4-amino-2,5-dimethylbenzenethiol; 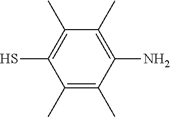
4-amino-2,3,5,6-tetramethylbenzenethiol; 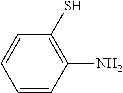
2-aminobenzenethiol (2-NH₂PLSH); 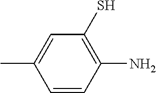
2-amino-5-methylbenzenethiol; 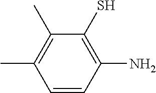
6-amino-2,3-dimethylbenzenethiol; 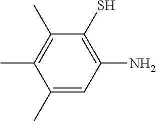
6-amino-2,3,4-trimethylbenzenethiol; and 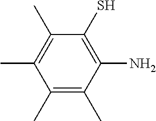
2-amino-3,4,5,6-tetramethylbenzenethiol.

8. The composition according to claim 1, wherein the diazirine of formula (II) is selected from the group consisting of: 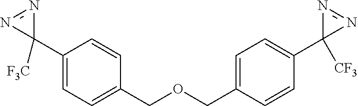 [SMILES: FC(F)(F)C1(c2ccc(COCc3ccc(C4(C(F)(F)F)N=N4)cc3)cc2)N=N1]
3,3′-((oxybis(methylene))bis(4,1-phenylene))bis(3-(trifluoromethyl)-3H-diazirine) (PCG-1); 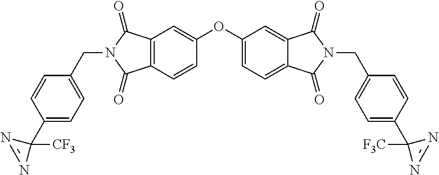 [SMILES: O=C1c2ccc(Oc3ccc4c(c3)C(=O)N(Cc3ccc(C5(C(F)(F)F)N=N5)cc3)C4=O)cc2C(=O)N1Cc1ccc(C2(C(F)(F)F)N=N2)cc1]
5,5′-oxybis(2-(4-(3-(trifluoromethyl)-3H-diazirin-3-yl)benzyl)isoindoline-1,3-dione) (PCG-3); 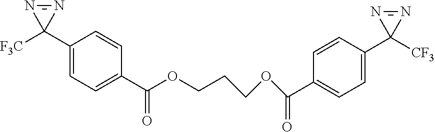 [SMILES: O=C(OCCCOC(=O)c1ccc(C2(C(F)(F)F)N=N2)cc1)c1ccc(C2(C(F)(F)F)N=N2)cc1]
propane-1,3-diyl bis(4-(3-(trifluoromethyl)-3H-diazirin-3-yl)benzoate); 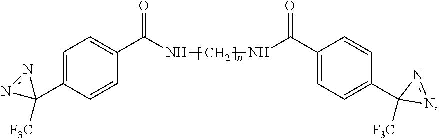
where n is 7 or 12; 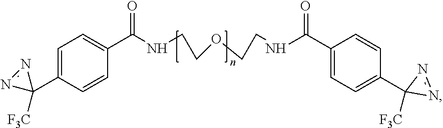
where n is 3 or 5; 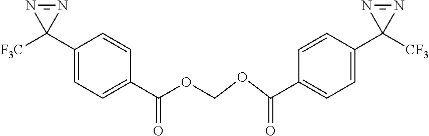 [SMILES: O=C(OCOC(=O)c1ccc(C2(C(F)(F)F)N=N2)cc1)c1ccc(C2(C(F)(F)F)N=N2)cc1]
methylene bis(4-(3-(trifluoromethyl)-3H-diazirin-3-yl)benzoate); 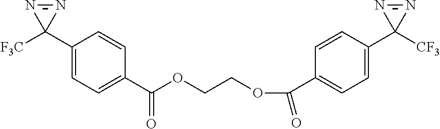 [SMILES: O=C(OCCOC(=O)c1ccc(C2(C(F)(F)F)N=N2)cc1)c1ccc(C2(C(F)(F)F)N=N2)cc1]
ethane-1,2-diyl bis(4-(3-(trifluoromethyl)-3H-diazirin-3-yl)benzoate); 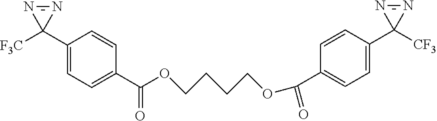 [SMILES: O=C(OCCCCOC(=O)c1ccc(C2(C(F)(F)F)N=N2)cc1)c1ccc(C2(C(F)(F)F)N=N2)cc1]
butane-1,4-diyl bis(4-(3-(trifluoromethyl)-3H-diazirin-3-yl)benzoate); 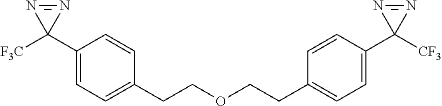 [SMILES: FC(F)(F)C1(c2ccc(CCOCCc3ccc(C4(C(F)(F)F)N=N4)cc3)cc2)N=N1]
3,3′-((oxybis(ethane-2,1-diyl))bis(4,1-phenylene))bis(3-(trifluoromethyl)-3H-diazirine); 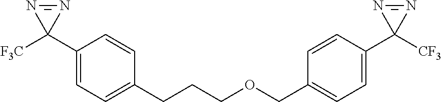 [SMILES: FC(F)(F)C1(c2ccc(CCCOCc3ccc(C4(C(F)(F)F)N=N4)cc3)cc2)N=N1]
3-(trifluoromethyl)-3-(4-(3-((4-(3-(trifluoromethyl)-3H-diazirin-3-yl)benzyl)oxy)propyl)phenyl)-3H-diazirine; 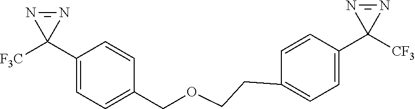 [SMILES: FC(F)(F)C1(c2ccc(CCOCc3ccc(C4(C(F)(F)F)N=N4)cc3)cc2)N=N1]
3-(trifluoromethyl)-3-(4-(2-((4-(3-(trifluoromethyl)-3H-diazirin-3-yl)benzyl)oxy)ethyl)phenyl)-3H-diazirine; 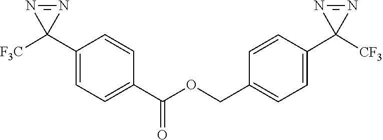 [SMILES: O=C(OCc1ccc(C2(C(F)(F)F)N=N2)cc1)c1ccc(C2(C(F)(F)F)N=N2)cc1]
4-(3-(trifluoromethyl)-3H-diazirin-3-yl)benzyl 4-(3-(trifluoromethyl)-3H-diazirin-3-yl)benzoate; 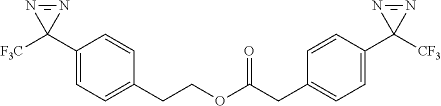 [SMILES: O=C(Cc1ccc(C2(C(F)(F)F)N=N2)cc1)OCCc1ccc(C2(C(F)(F)F)N=N2)cc1]
4-(3-(trifluoromethyl)-3H-diazirin-3-yl)phenethyl 2-(4-(3-(trifluoromethyl)-3H-diazirin-3-yl)phenyl)acetate; 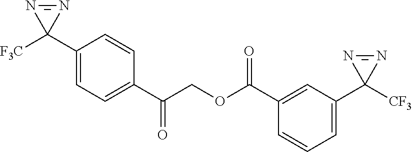 [SMILES: O=C(COC(=O)c1cccc(C2(C(F)(F)F)N=N2)c1)c1ccc(C2(C(F)(F)F)N=N2)cc1]
2-oxo-2-(4-(3-(trifluoromethyl)-3H-diazirin-3-yl)phenyl)ethyl 3-(3-(trifluoromethyl)-3H-diazirin-3-yl)benzoate; 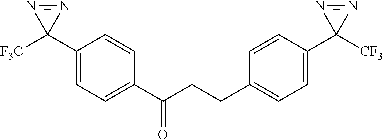 [SMILES: O=C(CCc1ccc(C2(C(F)(F)F)N=N2)cc1)c1ccc(C2(C(F)(F)F)N=N2)cc1]
1,3-bis(4-(3-(trifluoromethyl)-3H-diazirin-3-yl)phenyl)propan-1-one; 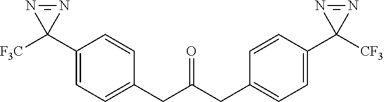 [SMILES: O=C(Cc1ccc(C2(C(F)(F)F)N=N2)cc1)Cc1ccc(C2(C(F)(F)F)N=N2)cc1]
1,3-bis(4-(3-(trifluoromethyl)-3H-diazirin-3-yl)phenyl)propan-2-one; 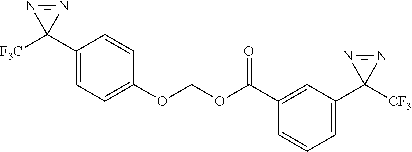 [SMILES: O=C(OCOc1ccc(C2(C(F)(F)F)N=N2)cc1)c1cccc(C2(C(F)(F)F)N=N2)c1]
(4-(3-(trifluoromethyl)-3H-diazirin-3-yl)phenoxy)methyl 3-(3-(trifluoromethyl)-3H-diazirin-3-yl)benzoate; 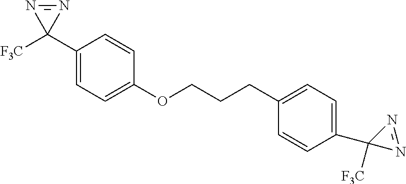 [SMILES: FC(F)(F)C1(c2ccc(CCCOc3ccc(C4(C(F)(F)F)N=N4)cc3)cc2)N=N1]
1,2-bis(4-(3-(trifluoromethyl)-3H-diazirin-3-yl)phenoxy)ethane; 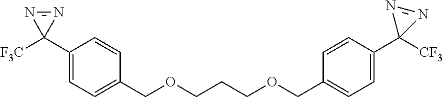 [SMILES: FC(F)(F)C1(c2ccc(COCCCOCc3ccc(C4(C(F)(F)F)N=N4)cc3)cc2)N=N1]
1,3-bis((4-(3-(trifluoromethyl)-3H-diazirin-3-yl)benzyl)oxy)propane; 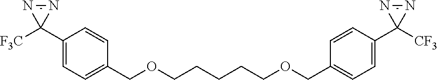 [SMILES: FC(F)(F)C1(c2ccc(COCCCCCOCc3ccc(C4(C(F)(F)F)N=N4)cc3)cc2)N=N1]
1,5-bis((4-(3-(trifluoromethyl)-3H-diazirin-3-yl)benzyl)oxy)pentane; 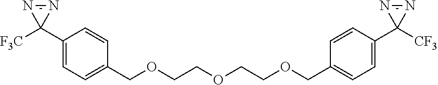 [SMILES: FC(F)(F)C1(c2ccc(COCCOCCOCc3ccc(C4(C(F)(F)F)N=N4)cc3)cc2)N=N1]
3,3′-((((oxybis(ethane-2,1-diyl))bis(oxy))bis(methylene))bis(4,1-phenylene))bis(3-(trifluoromethyl)-3H-diazirine); 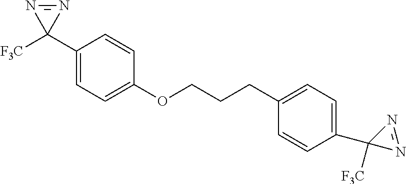 [SMILES: FC(F)(F)C1(c2ccc(CCCOc3ccc(C4(C(F)(F)F)N=N4)cc3)cc2)N=N1]
3-(trifluoromethyl)-3-(4-(3-(4-(3-(trifluoromethyl)-3H-diazirin-3-yl)phenoxy)propyl)phenyl)-3H-diazirine; 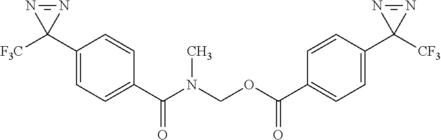 [SMILES: CN(COC(=O)c1ccc(C2(C(F)(F)F)N=N2)cc1)C(=O)c1ccc(C2(C(F)(F)F)N=N2)cc1]
(N-methyl-4-(3-(trifluoromethyl)-3H-diazirin-3-yl)benzamido)methyl 4-(3-(trifluoromethyl)-3H-diazirin-3-yl)benzoate; and 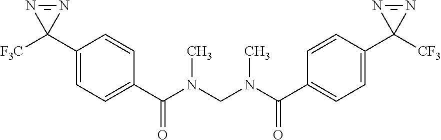 [SMILES: CN(CN(C)C(=O)c1ccc(C2(C(F)(F)F)N=N2)cc1)C(=O)c1ccc(C2(C(F)(F)F)N=N2)cc1]
N,N′-methylenebis(N-methyl-4-(3-(trifluoromethyl)-3H-diazirin-3-yl)benzamide).

9. The composition according to claim 1, which is selected from the group consisting of:
a polyamic acid formed from 1H,3H-benzo[1,2-c:4,5-c′]difuran-1,3,5,7-tetraone (PMDA), 4,4′-(1,3-phenylenebis(oxy))dianiline (APB), a diamine of formula (ID) (JD230) and 3,3′-((oxybis(methylene))bis(4,1-phenylene))bis(3-(trifluoromethyl)-3H-diazirine) (PCG-1);
a polyamic acid formed from 5,5′-(perfluoropropane-2,2-diyl)bis(isobenzofuran-1,3-dione) (6FDA), 1H,3H-benzo[1,2-c:4,5-c′]difuran-1,3,5,7-tetraone (PMDA), 4,4′-([1,1′-biphenyl]-4,4′-diylbis(oxy))bis(3-(trifluoromethyl)aniline) (6BF), 2,2′-bis(trifluoromethyl)-[1,1′-biphenyl]-4,4′-diamine (PFMB) and 3,3′-((oxybis(methylene))bis(4,1-phenylene))bis(3-(trifluoromethyl)-3H-diazirine) (PCG-1):
a polyamic acid formed from 5,5′-(perfluoropropane-2,2-diyl)bis(isobenzofuran-1,3-dione) (6FDA), 1H,3H-benzo[1,2-c:4,5-c′]difuran-1,3,5,7-tetraone (PMDA), 4,4′-([1,1′-biphenyl]-4,4′-diylbis(oxy))bis(3-(trifluoromethyl)aniline) (6BF), 2,2′-bis(trifluoromethyl)-[1,1′-biphenyl]-4,4′-diamine (PFMB), 1-(2-aminoethyl)-3,4-dimethyl-1H-pyrrole-2,5-dione (DMMIEtNH₂) and 3,3′-((oxybis(methylene))bis(4, 1-phenylene))bis(3-(trifluoromethyl)-3H-diazirine) (PCG-1):
a polyamic acid formed from 5,5′-(perfluoropropane-2,2-diyl)bis(isobenzofuran-1,3-dione) (6FDA), 1H,3H-benzo[1,2-c:4,5-c′]difuran-1,3,5,7-tetraone (PMDA), 4,4′-([1,1′-biphenyl]-4,4′-diylbis(oxy))bis(3-(trifluoromethyl)aniline) (6BF), 2,2′-bis(trifluoromethyl)-[1,1′-biphenyl]-4,4′-diamine (PFMB), 1-(2-aminoethyl)-3,4-dimethyl-1H-pyrrole-2,5-dione (DMMIEtNH₂) and 5,5′-oxybis(2-(4-(3-(trifluoromethyl)-3H-diazirin-3-yl)benzyl)isoindoline-1,3-dione) (PCG-3);
a polyamic acid formed from 5,5′-(perfluoropropane-2,2-diyl)bis(isobenzofuran-1,3-dione) (6FDA), 1H,3H-benzo[1,2-c:4,5-c′]difuran-1,3,5,7-tetraone (PMDA), 4,4′-([1,1′-biphenyl]-4,4′-diylbis(oxy))bis(3-(trifluoromethyl)aniline) (6BF), 2,2′-bis(trifluoromethyl)-[1,1′-biphenyl]-4,4′-diamine (PFMB), 2-aminobenzenethiol (2-NH₂PhSH) and 3,3′-((oxybis(methylene))bis(4,1-phenylene))bis(3-(trifluoromethyl)-3H-diazirine) (PCG-1); and
a polyamic acid formed from 5,5′-(perfluoropropane-2,2-diyl)bis(isobenzofuran-1,3-dione) (6FDA), 1H,3H-benzo[1,2-c:4,5-c′]difuran-1,3,5,7-tetraone (PMDA), 4,4′-([1,1′-biphenyl]-4,4′-diylbis(oxy))bis(3-(trifluoromethyl)aniline) (6BF), 4,4′-methylenebis(2,6-dimethylaniline) (DO3), 1-(2-aminoethyl)-3,4-dimethyl-1H-pyrrole-2,5-dione (DMMIEtNH₂) and 3,3′-((oxybis(methylene))bis(4,1-phenylene))bis(3-(trifluoromethyl)-3H-diazirine) (PCG-1); and
a polyamic acid formed from 5,5′-(perfluoropropane-2,2-diyl)bis(isobenzofuran-1,3-dione) (6FDA), 1H,3H-benzo[1,2-c:4,5-c′]difuran-1,3,5,7-tetraone (PMDA), 4,4′-([1,1′-biphenyl]-4,4′-diylbis(oxy))bis(3-(trifluoromethyl)aniline) (6BF), 4,4′-methylenebis(2,6-dimethylaniline) (DO3), 1-(2-aminoethyl)-3,4-dimethyl-1H-pyrrole-2,5-dione (DMMIEtNH₂) and 5,5′-oxybis(2-(4-(3-(trifluoromethyl)-3H-diazirin-3-yl)benzyl)isoindoline-1,3-dione) (PCG-3).

10. The composition according to claim 1, wherein the polyamic acid is having a weight average molecular weight (M_{w}) of at least 20,000 and is soluble in an organic solvent.

11. The composition according to claim 10, wherein the organic solvent is selected from the group consisting of N-methyl-2-pyrrolidone (NMP), γ-butyrolactone (GBL), N,N-dimethylacetamide (DMAc), propylene glycol monomethyl ether acetate (PGMEA), dimethyl sulfoxide (DMSO), cyclopentanone, cyclohexanone, 2-butanone and 2-heptanone and mixtures in any combination thereof.

12. The composition according to claim 1, further comprising one or more epoxy crosslinking agents selected from the group consisting of: 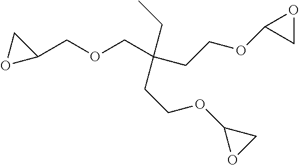 [SMILES: CCC(CCOC1CO1)(CCOC1CO1)COCC1CO1]
2,2′-(((2-ethyl-2-((oxiran-2-ylmethoxy)methyl)propane-1,3-diyl)bis(oxy))bis(methylene))bis(oxirane) (TMPTGE); 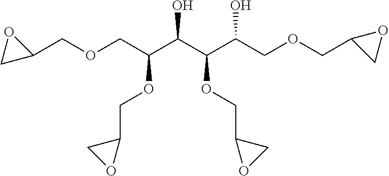 [SMILES: O[C@@H]([C@H](OCC1CO1)[C@H](O)COCC1CO1)[C@H](COCC1CO1)OCC1CO1]
(2R,3R,4R,5S)-1,3,5,6-tetrakis(oxiran-2-ylmethoxy)hexane-2,4-diol (also known as tetrakis-O)-(oxiranylmethyl)-D-glucitol) (Denacol EX-614 from Nagase); 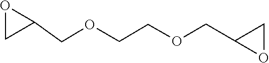 [SMILES: C(COCC1CO1)OCC1CO1]
1,2-bis(oxiran-2-ylmethoxy)ethane;
a compound of formula (V): 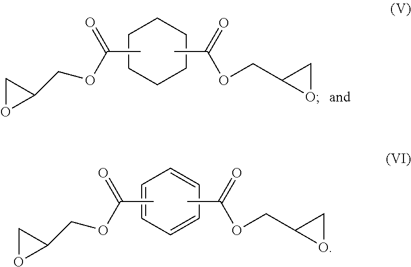
a compound of formula (VI):

13. A composition comprising:
a) a polyamic acid selected from the group consisting of a polyamic acid of formula (IA), an end capped polyamic acid of formula (IB) and an end capped polyamic acid of formula (IC): 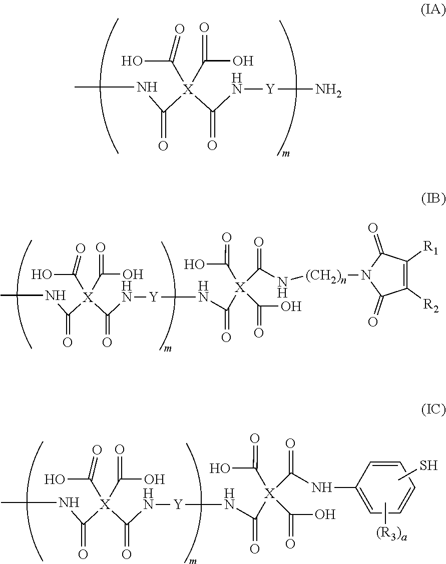
wherein:
m is an integer of at least 50;
n is an integer from 1 to 12, inclusive;
a is an integer from 1 to 4;
X is one or more distinct tetravalent organic group, which is derived from one or more dianhydrides selected from the group consisting of: 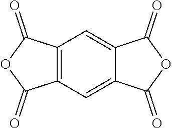 [SMILES: O=c1oc(=O)c2cc3c(=O)oc(=O)c3cc12]
1H,3H-benzo[1,2-c:4,5-c′]difuran-1,3,5,7-tetraone (PMDA); 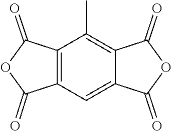 [SMILES: Cc1c2c(=O)oc(=O)c2cc2c(=O)oc(=O)c12]
4-methyl-1H,3H-benzo[1,2-c:4,5-c′]difuran-1,3,5,7-tetraone; 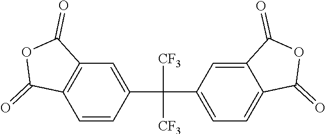 [SMILES: O=C1OC(=O)c2cc(C(c3ccc4c(c3)C(=O)OC4=O)(C(F)(F)F)C(F)(F)F)ccc21]
5,5′-(perfluoropropane-2,2-diyl)bis(isobenzofuran-1,3-dione) (6FDA); 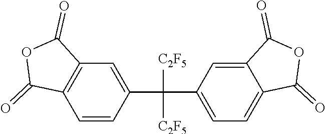
5,5′-(perfluoropentane-3,3-diyl)bis(isobenzofuran-1,3-dione); 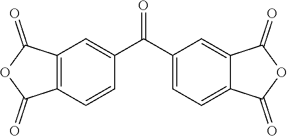 [SMILES: O=C(c1ccc2c(c1)C(=O)OC2=O)c1ccc2c(c1)C(=O)OC2=O]
5,5′-carbonylbis(isobenzofuran-1,3-dione) (BTDA); 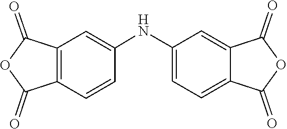 [SMILES: O=C1OC(=O)c2cc(Nc3ccc4c(c3)C(=O)OC4=O)ccc21]
5,5′-azanediylbis(isobenzofuran-1,3-dione); 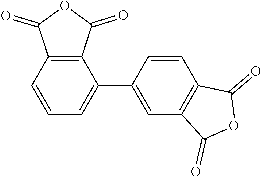 [SMILES: O=C1OC(=O)c2cc(-c3cccc4c3C(=O)OC4=O)ccc21]
[4,5′-biisobenzofuran]-1,1′,3,3′-tetraone (α-BPDA); 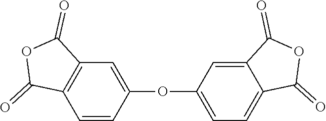 [SMILES: O=C1OC(=O)c2cc(Oc3ccc4c(c3)C(=O)OC4=O)ccc21]
5,5′-oxybis(isobenzofuran-1,3-dione) (ODPA); 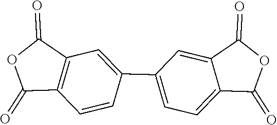 [SMILES: O=C1OC(=O)c2cc(-c3ccc4c(c3)C(=O)OC4=O)ccc21]
[5,5′-biisobenzofuran]-1,1′,3,3′-tetraone (BPDA); and 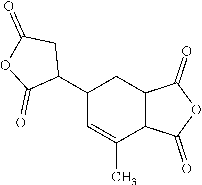 [SMILES: CC1=CC(C2CC(=O)OC2=O)CC2C(=O)OC(=O)C12]
5-(2,5-dioxotetrahydrofuran-3-yl)-7-methyl-3a,4,5,7a-tetrahydroisobenzofuran-1,3-dione (D1901);
Y is one or more distinct divalent organic group derived from a diamine; and
R₁ and R₂ are the same or different and each independently of one another selected from the group consisting of hydrogen, linear or branched (C₁-C₁₆)alkyl, perfluoro(C₁-C₁₂)alkyl, linear or branched (C₁-C₁₆)alkenyl, perfluoro(C₁-C₁₂)alkenyl, (C₆-C₁₀)aryl and (C₆-C₁₀)aryl(C₁-C₃)alkyl; or
R₁ and R₂ taken together with the carbon atoms to which they are attached to form a 5 to 7 membered monocyclic ring, a 6 to 12 membered bicyclic ring or a 9 to 14 membered tricyclic ring, said rings optionally containing one or more heteroatoms selected from O, N and S, and said rings optionally substituted with one or more groups selected from the group consisting of methyl, ethyl, linear or branched (C₃-C₈)alkyl, (C₆-C₁₀)aryl, halogen, hydroxy, linear or branched (C₁-C₈)alkoxy and (C₆-C₁₀)aryloxy; and
each of R₃ is independently selected from the group consisting of hydrogen, halogen, hydroxy, methyl, ethyl, linear or branched (C₃-C₆)alkyl, trifluoromethyl, pentafluoroethyl, linear or branched perfluoro(C₃-C₆)alkyl, methoxy, ethoxy, linear or branched (C₃-C₆)alkyloxy, (C₂-C₆)acyl, (C₂-C₆)acyloxy, phenyl and phenoxy; and
b) a diazirine compound of formula (II): 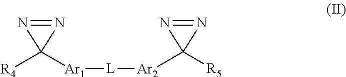
wherein,
L is a divalent linking or a spacer group selected from the group consisting of:
—C(O)O—R₆—OC(O)—, —C(O)O—R₆—, —R₆—OC(O)—R₆—, —C(O)—R₆—OC(O)—, —C(O)—R₆—, —R₆—C(O)—R₆—, —O—R₆—OC(O)—, —O—R₆—O—, —O—R₆—, —R₆—O—R₆—, —C(O)NH—(CH₂)_{b}—NH(CO)—, where b is an integer from 1 to 15, inclusive, —C(O)NH—(CH₂CH₂O)_{c}(CH₂)ₐ—NR₅(CO)—, where c is an integer from 2 to 6, inclusive and d is an integer from 1 to 6, inclusive, and each occurrence of R₆ may be the same or different which is a divalent group independently selected from the group consisting of methyl, ethyl, linear or branched (C₃-C₁₂)alkyl, (C₃-C₁₂)cycloalkyl, (C₆-C₁₂)aryl, (C₆-C₁₂)aryl(C₁-C₁₂)alkyl, (C₆-C₁₀)heteroaryl, (C₆-C₁₀)heteroaryl(C₁-C₁₂)alkyl, —(CH₂—CH₂—O)ₐ—, where a is an integer from 1 to 10, inclusive;
R₄ and R₅ are the same or different and each independently selected from the group consisting of methyl, ethyl, linear or branched (C₃-C₁₂)alkyl, where portions of hydrogen on alkyl are replaced with fluorine, (C₁-C₁₂)perfluoroalkyl, (C₆-C₁₂)aryl, (C₆-C₁₂)aryl(C₁-C₁₂)alkyl, where portions of hydrogen on alkyl are replaced with fluorine, and (C₆-C₁₂)arylperfluoro(C₁-C₁₂)alkyl; and
Ar₁ and Ar₂ are the same or different and each is independently selected from (C₆-C₁₂)arylene or (C₆-C₁₂)heteroarylene group optionally substituted with a group selected from the group consisting of halogen, —OH, methyl, ethyl, linear or branched (C₃-C₆)alkyl, (C₁-C₄)alkoxy, (C₆-C₁₀)aryl, (C₆-C₁₂)aryloxy, (C₆-C₁₂)aryl(C₁-C₄)alkyl and (C₆-C₁₂)aryl(C₁-C₄)alkyloxy; and
c) one or more epoxy crosslinking agents.

14. The composition according to claim 13, wherein the epoxy crosslinking agent is selected from the group consisting of: 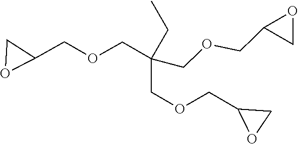 [SMILES: CCC(COCC1CO1)(COCC1CO1)COCC1CO1]
2,2′-(((2-ethyl-2-((oxiran-2-ylmethoxy)methyl)propane-1,3-diyl)bis(oxy))bis(methylene))bis(oxirane) (TMPTGE); 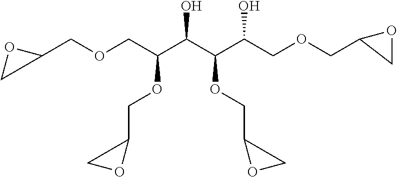 [SMILES: O[C@@H]([C@H](OCC1CO1)[C@H](O)COCC1CO1)[C@H](COCC1CO1)OCC1CO1]
(2R,3R,4R,5S)-1,3,5,6-tetrakis(oxiran-2-ylmethoxy)hexane-2,4-diol (also known as tetrakis-O—(oxiranylmethyl)-D-glucitol) (Denacol EX-614 from Nagase); 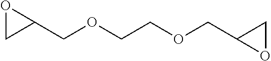 [SMILES: C(COCC1CO1)OCC1CO1]
1,2-bis(oxiran-2-ylmethoxy)ethane;
a compound of formula (V): 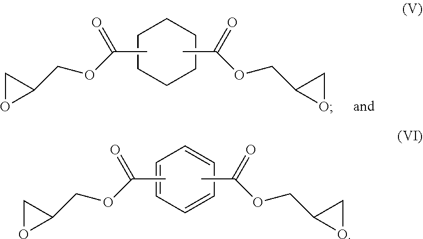
a compound of formula (VI):

15. A cured product comprising the composition of claim 1.

16. A cured product comprising the composition of claim 13.

17. A microelectronic or optoelectronic device comprising one or more of a redistribution layer (RDL) structure, a chip-stack structure, a CMOS image sensor dam structure, where said structures comprising a composition according to claim 1.

18. A microelectronic or optoelectronic device comprising one or more of a redistribution layer (RDL) structure, a chip-stack structure, a CMOS image sensor dam structure, where said structures comprising a composition according to claim 13.

19. A diazirine of formula (IIA): 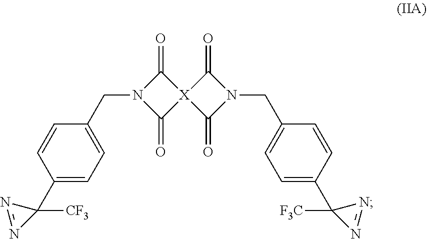
wherein:
X is a tetravalent organic group, which is derived from a dianhydride selected from the group consisting of: 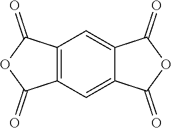 [SMILES: O=c1oc(=O)c2cc3c(=O)oc(=O)c3cc12]
1H,3H-benzo[1,2-c:4,5-c′]difuran-1,3,5,7-tetraone (PMDA); 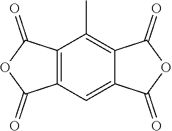 [SMILES: Cc1c2c(=O)oc(=O)c2cc2c(=O)oc(=O)c12]
4-methyl-1H,3H-benzo[1,2-c:4,5-c′]difuran-1,3,5,7-tetraone; 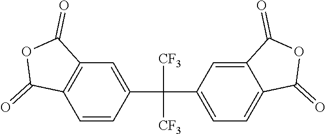 [SMILES: O=C1OC(=O)c2cc(C(c3ccc4c(c3)C(=O)OC4=O)(C(F)(F)F)C(F)(F)F)ccc21]
5,5′-(perfluoropropane-2,2-diyl)bis(isobenzofuran-1,3-dione) (6FDA); 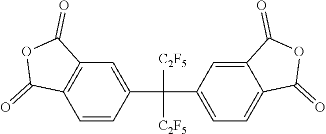
5,5′-(perfluoropentane-3,3-diyl)bis(isobenzofuran-1,3-dione); 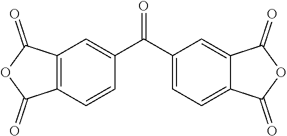 [SMILES: O=C(c1ccc2c(c1)C(=O)OC2=O)c1ccc2c(c1)C(=O)OC2=O]
5,5′-carbonylbis(isobenzofuran-1,3-dione) (BTDA); 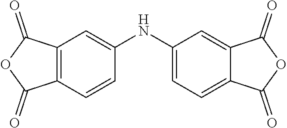 [SMILES: O=C1OC(=O)c2cc(Nc3ccc4c(c3)C(=O)OC4=O)ccc21]
5,5′-azanediylbis(isobenzofuran-1,3-dione); 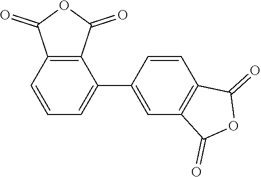 [SMILES: O=C1OC(=O)c2cc(-c3cccc4c3C(=O)OC4=O)ccc21]
[4,5′-biisobenzofuran]-1,1′,3,3′-tetraone (α-BPDA); 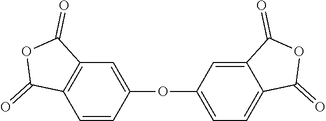 [SMILES: O=C1OC(=O)c2cc(Oc3ccc4c(c3)C(=O)OC4=O)ccc21]
5,5′-oxybis(isobenzofuran-1,3-dione) (ODPA); 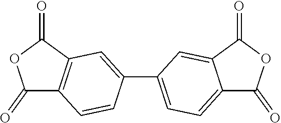 [SMILES: O=C1OC(=O)c2cc(-c3ccc4c(c3)C(=O)OC4=O)ccc21]
[5,5′-biisobenzofuran]-1,1′,3,3′-tetraone (BPDA); and 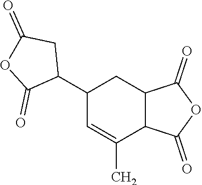 [SMILES: CC1=CC(C2CC(=O)OC2=O)CC2C(=O)OC(=O)C12]
5-(2,5-dioxotetrahydrofuran-3-yl)-7-methyl-3a,4,5,7a-tetrahydroisobenzofuran-1,3-dione.

20. The diazirine of formula (IIA) according to claim 19, which is: 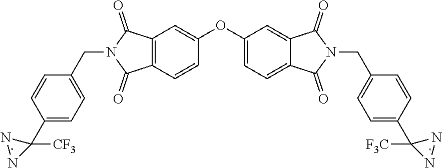 [SMILES: O=C1c2ccc(Oc3ccc4c(c3)C(=O)N(Cc3ccc(C5(C(F)(F)F)N=N5)cc3)C4=O)cc2C(=O)N1Cc1ccc(C2(C(F)(F)F)N=N2)cc1]
5,5′-oxybis(2-(4-(3-(trifluoromethyl)-3H-diazirin-3-yl)benzyl)isoindoline-1,3-dione).
